(19) United States
(12) Patent Application Publication
(10) Pub. No.: US 2016/0313319 A1
(43) Pub. Date: Oct. 27, 2016

(54) DIAGNOSTIC TEST STRIPS WITH MULTIPLE LAMINATED LAYERS CONTAINING ONE OR MORE REAGENT-CARRYING PADS IN ONE OR MORE LAYERS

(71) Applicants: Ted Titmus, Mission Viejo, CA (US); William Pat Price, Henderson, NV (US)

(72) Inventors: Ted Titmus, Mission Viejo, CA (US); William Pat Price, Henderson, NV (US)

(21) Appl. No.: 14/694,779

(22) Filed: Apr. 23, 2015

(51) Int. Cl. G01N 33/543

(52) U.S. Cl. CPC G01N 33/54386

(57) ABSTRACT

Some embodiments of the disclosure provide a diagnostic assay test strip for detecting analytes on one or more test pad using one or more reagents. The diagnostic test strip may include a supporting strip having one or more indentations and one or more test pads capable of fitting in those indentations. The diagnostic test strip may include one or more test pads on one or more sides of a carrier strip. The diagnostic test strip may include one or more intermediate layers and a carrier layer and a bottom laminate layer where the test pads are interspersed on the carrier layer. The diagnostic test strip may include test pads with a top, bottom, and sides, where the top and bottom of the pads have a trailing edge and a leading edge that are mechanically fixed to the carrier strip.

### BACKGROUND OF THE INVENTION

1. Field of the Invention

The invention generally relates to diagnostic assay materials. More specifically, the invention relates to diagnostic test strips having one or more test pads, each of which has one or more layers, and methods for the use of said diagnostic test strips.

2. Description of the Related Art

Many types of assays have been used to detect the presence of various substances, generally referred to as analytes, in physiological fluids such as urine and blood. These assays often involve antigen-antibody reactions; synthetic conjugates comprising radioactive, enzymatic, fluorescent, or visually observable metal sol tags; and specially designed reactor chambers. In all these assays, there is a receptor; e.g., an antibody or chemical, which is specific for the selected analyte; and a means for detecting the presence, and often the amount, of the analyte. While some tests are designed to make a quantitative determination, in many circumstances all that is required is a qualitative positive/negative indication. However, in some circumstances the analyte of interest is present in the test sample in very small concentrations. Such circumstances require an assay to be very sensitive in order to detect the presence, absence, and/or concentration of the desired analyte. False positives and false negatives for qualitative assays can also be especially problematic.

Unlike other forms of fluid specimens, the collection of oral fluid, such as saliva, for diagnostic purposes is complicated by many factors. These factors include the low volumes of salivary fluid secreted into the oral cavity, the relatively high viscosity of salivary fluid, and the diverse anatomic dispersion of the salivary glands. Moreover, many devices utilize surface tension, cohesion, adhesion, wicking, and/or capillary action to create lateral flow to contact the test sample with the test area. Such devices require substantial amounts of a liquid sample to provide lateral flow, yet many samples, such as saliva, have low and/or limited volumes. Because of these factors, the testing of salivary specimens has not been extensively developed. However, it is known that human saliva contains some of the same materials that may also be present in a human's blood. For example, it is known that human saliva carries lymphocytes, plasma cells and immunoglobulins that are directly related to the immunoglobulins found in the blood. In addition, saliva carries immunoglobulins that are believed to be peculiar to saliva, for example, the antibody known as secretory IgA. Because of the association between immunoglobulins of the blood and saliva, as well as the occurrence of secretory IgA, antigen-antibody tests may prove useful for conducting diagnostic assays with saliva, amongst other forms of physiological fluids.

Many technological fields and applications require rapid, accurate, and reproducible analyte detection. This is especially true for law enforcement officials, physicians, aid workers, employers, parents, and other assay users because safety and livelihood could depend upon the presence, absence, and/or concentration of certain analytes. In some circumstances, untrained individuals may need to test for the presence of an analyte to determine personal safety and health. However, common techniques for collecting and testing specimens are often complicated or invasive. For example, collecting salivary fluid often involves the use of capillary tubes, micropipette suctioning, chewing on paraffin or foam, and/or aspiration from the mouth into polypropylene syringes. Such techniques inevitably complicate the collection of salivary specimens and increase the likelihood of user error and/or false negatives. Additionally, contamination of the test sample can be a significant cause of false positives and false negatives. Because the assays are exposed to the environment before and after the test fluid is applied to the assays, environmental contaminants may affect results of the assay. What is needed is a simple, accurate assay that provides trustworthy signaling of the presence, absence, and/or concentration of one or more analytes in a given sample. These and other objects and features of the invention will be apparent from the following description, drawings, and claims.

### SUMMARY OF THE INVENTION

Embodiments described herein are directed to diagnostic test strips and methods of using the same. From this description, in conjunction with other items, the advantages of the invention will become clear and apparent more so based upon the hereinafter descriptions and claims, which are supported by drawings with numbers relating to parts, wherein are described in the following sections containing the relating numbers.

Some embodiments provide for a diagnostic test strip for detecting analytes having an active reference zone that contains a carrier strip and at least one test pad. Such test pads contain at least one, but preferably at least two transparent membranes as test pad layers. A first transparent membrane has a test reagent that indicates the presence of at least one reference analyte while a second transparent membrane has a test reagent that indicates the presence of at least one target analyte. The test reagents are arranged on the membranes in a substantially single striped shape, and the transparent membranes are opposed to each other such that the striped shapes are at substantially right angles. In other embodiments, the test reagents are arranged in a substantially single striped shape on a portion of the transparent membranes, and the transparent membranes are opposed to each other such that detecting both the reference analyte and the target analyte produces a signal in various shapes, such as a circle, oval, square, plus sign, an “X” sign, and/or a checkmark. Optionally, transparent membranes may be opposed with test reagents such that the detection of both a target analyte and a reference analyte produces a signal within a signal. Furthermore, a single membrane may contain two or more separate test reagents for target analytes, each reagent disposed at substantially right angles to any test reagent for the reference analyte on another membrane.

In one embodiment, a diagnostic assay test strip is provided. The test strip includes one or more indentations wherein the each of the indentations contains at least one test pad, the test pad having a top and a bottom surface, wherein the bottom surface in contact with the test strip. Advantageously, the test pad contains one or more test reagents. Each of the one or more indentations may extend across the width of the test strip. In one embodiment, there is a single indentation. In another embodiment, there are two indentations. The indentation may extend for substantially the entire width of the test strip. The top surface of the test pad may be flush with the surface of the test strip or may be below the surface of the test strip. Optionally, the indentations are sealed with a removable cover.

In another embodiment, a diagnostic test strip having a carrier strip and at least two test pads is described. At least one test pad may be disposed on the opposing side of the carrier strip from at least one other test pad and the test pads may extend above the surface of the carrier strip. Advantageously, at least one test pad contains a test reagent. In certain aspects, the at least two test pads may contain test reagents and the test reagents in each pad may be the same or different. Optionally, the test reagents each test for a different marker on the same analyte. At least one test pad may contain a signaling reagent.

The diagnostic test strip may either be substantially non-porous or substantially porous.

In yet another aspect of the invention, the test strip further includes one or more additional test pads disposed on the opposing or adjacent sides of the carrier strip. The test pads may be substantially porous. Alternatively, the test pads may be substantially non-porous.

In one aspect, the test strip includes at least two test pads which extend for substantially the entire width of the side of the strip to which they are disposed.

Advantageously, the test pad has a test reagent that tests for an analyte found in saliva, serum, plasma, urine, blood, semen, sputum, ascites, or cerebral spinal fluid. The at least one test pad may have a signal reagent for an analyte.

Also provided herein is a method of detecting one or more analytes in a patient sample. The method includes a) contacting a test strip as described above with a patient sample such that the test pads come in contact with the sample; and reading the test result from the one or more test pads. The method may further include adding one or more signaling reagents to the test strip following the addition of the patient sample such that the reagents contacts the test pads.

The patient sample may be saliva. The test strip may be contacted with the patient's tongue. In other embodiments, the patient sample is serum, semen, or blood. When the sample is blood, the test strip may be directly contacted with the source of the patient's blood. The sample may be plasma, ascites, sputum, or spinal fluid.

In another embodiment, a diagnostic test strip is provided, wherein the test strip includes a test pad carrier, one or more test pads, where the test pads are interspersed, and interrupt, the test pad carrier, an intermediate laminate layer, where the intermediate layer is longer than the test pad carrier, and the test pad carrier and test pads are disposed on top of the intermediate layer, and a bottom laminate layer, where the bottom of the intermediate layer is disposed on top of the bottom laminate layer, wherein at least one of the test pads contains a test reagent. The top of the test pad carrier and the one or more test pads may be substantially flush. In another embodiment, the one or more test pads extends above the top of the test pad carrier.

In one embodiment, there is one test pad. The one test pad may be located at one end of the test strip. It will also be appreciated that there may be at least two test pads. Each test pad may contain the same or a different test reagent. The test reagent may be a signal generating reagent. Advantageously, the signal generating reagent is present on at least one test pad. The test pad may contain at least one test reagent. The two test pads may be separated by a length of test carrier pad. There may be more than one test pad and one test pad may not contain a test reagent. The diagnostic test strip may include at least one test pad which contains a reagent that tests for an analyte present in mammalian saliva or mammalian urine.

Optionally, the at least one test pad may contain a reagent that test for an analyte present in plasma, serum, blood, ascites, cerebral spinal fluid, semen, or sputum.

In one embodiment, there are more than one test pads and each one detects a different marker on the same analyte.

A method for detecting one or more analytes in a patient sample is provided. The method includes applying the sample on a test strip so that it contacts the one or more test pads present on the test strip; and reading the test result from the one or more test pads. The method can optionally include removing any removable covers present on the test strip before applying the sample. The patient sample applied may be saliva and the sample may be applied by contacting the test strip with the patient's tongue. The method may further include applying a signal developing reagent to the test strip prior to reading the test result. The patient sample may be urine and may be collected by applying the urine directly to the test strip. The patient sample may be plasma, serum, or blood. In the case of blood, the test strip can be directly contacted with the source of the blood sample from the patient. The sample may be semen, ascites, sputum, or cerebral spinal fluid. Optionally, least one signaling reagent is added to the test strip such that it contacts the one or more test pads.

In one aspect of the invention, a diagnostic test strip is provided. The test strip includes a top, bottom and sides. Also included is at least one test pad comprised of a top, bottom and sides. Advantageously, the top and the bottom of the pads have a trailing edge and a leading edge. The trailing edge and leading edge may be mechanically fixed to the test strip. Optionally, the test pads are fixed to the test strip with staples.

In another aspect of the invention, one or more of a first group of staples bridges the trailing edge of the test pad and the test strip. An at least one second group of staples bridges the leading edge of the test pad and the test strip. In one embodiment, one staple each bridges the trailing and leading edges of the test pads and the test strip.

In still another aspect, the diagnostic test strip may include one or more of a third group of staples which bridges one side of the test pad and the test strip and one or more staples of a fourth group of staples bridges the side of test pad and the side and the top of the test strip.

Advantageously, in certain aspects of the invention, there may be at last two or more test pads, at least one of which is attached to the top of the test strip and at least one is attached to the bottom of the test strip. In another aspect, there are at least two or more test pads and at least one is attached to the top of the test strip and at least one other is attached to a side of the test strip.

In further aspects, the diagnostic test strip may include test pads, each test pads containing a reagent. Optionally, at least one of the test pads includes a signaling reagent. The reagent may be the same reagent for each test pad or, alternatively, each of the test pads may contain a different test reagent. Each of the reagents may detect a different marker on the same analyte.

In another aspect, the diagnostic test strip may include a single test pad mechanically fixed to the top of the pad. Alternatively, the test strip may include two test pads are mechanically fixed to the top of the pad. In still another aspect, the diagnostic test strip may comprise three test pads mechanically fixed to the top of the pad.

The location of the test pad may vary. In one embodiment, at least one test pad is mechanically fixed to the top of the strip and at least one test pad is attached test pad to the bottom of the strip. In another embodiment, one test pad may be mechanically attached to the top of the test strip and one mechanically attached to a side of the test strip.

The staples of the test strip may be metal, paper, or plastic.

In one aspect of the invention, the diagnostic test has indentations in the top of the strip and the test pads are fixed to the strip in these indentations. The indentations may contain the entire depth of the test pad such that the top of the test pad is flush with the top of the test strip. Alternatively, the indentations may be such that the top of the test pads are below the top of the test strip.

In certain aspects, the test pads of the diagnostic test extend substantially from side to side on the top of the test strip. The test strip may be substantially porous. Alternatively, the test strip may be substantially non-porous. Similarly, the test pads may be substantially non-porous.

The at least one test pad may include a reagent that tests for a saliva-borne analyte, sputum-borne analyte, serum-borne analyte, plasma-borne analyte, blood-borne analyte, urine-borne analyte, semen-borne analyte, cerebral spinal fluid-borne analyte, or ascites-borne analyte.

Also provided herein is a method for detecting analytes in a patient sample. The method may include contacting a test strip as described above with a patient sample so that the sample contacts the one or more test pads; and reading the results from the test strip. The method may further include contacting the test strip with one or more signaling reagents such that the one or more signaling reagents contacts the one or more test pads. The patient sample may be serum, semen, or urine. In the case of urine, the test strip may be directly contacted with the patient's urine stream. The patient sample may be saliva and the test strip may be contacted with the patient's tongue. The patient sample may be blood and the test strip may be contacted directly with the source of blood. The patient sample may also be ascites, sputum, or cerebral spinal fluid.

Some embodiments may have a single test pad, while other embodiments may have two or more test pads. A test pad may detect the same, or optionally different, target analytes. Moreover, two or more test pads may detect different markers on the same analyte. Two or more test pads may touch each other on the carrier strip, or two more test pads may optionally be separated on the carrier strip. Moreover, two of the two or more test pads may be on opposite sides of the carrier strip. Any test pad may be substantially covered in an oxygen-impermeable water-soluble membrane. Furthermore, one or more test pads may be in direct fluid contact with each other, and test pads may be in fluid contact with the carrier strip.

In some embodiments, the reference analyte and the target analyte are optionally found in patient samples such as saliva, sputum, blood serum, blood plasma, blood, urine, semen, ascites, cerebral spinal fluid, and/or fecal matter. The reference analyte may be alpha-amylase while the target analyte may optionally be any one or more drugs of abuse and/or therapeutic drugs.

Other embodiments provide for a method of detecting one or more analytes in a patient sample by contacting one or more test pads of an embodiment of a diagnostic test strip with a patient sample and reading the results from the embodiment. Patient samples may optionally be serum, semen, urine, saliva, blood, ascites, sputum, cerebral spinal fluid, and/or fecal material. Moreover, embodiments may be directly contacted with a patient's urine stream, source of bleeding, and/or tongue. Optionally, signaling reagents may be applied to one or more test pads of an embodiment of a diagnostic test strip.

### BRIEF DESCRIPTION OF THE DRAWINGS

FIG. 1A is a cross-sectional view of an embodiment of a diagnostic test strip having a test pad in an indentation in the carrier strip.

FIG. 1B is a top view of an embodiment of a diagnostic test strip having a test pad in an indentation in the carrier strip.

FIG. 1C is a perspective view of an embodiment of a diagnostic test strip having a test pad in an indentation in the carrier strip.

FIG. 2A is a cross-sectional view of an embodiment of a diagnostic test strip having two test pads in indentations in a carrier strip.

FIG. 2B is a top view of an embodiment of a diagnostic test strip having two test pads in indentations in a carrier strip.

FIG. 2C is a perspective view of an embodiment of a diagnostic test strip having two test pads in indentations in a carrier strip.

FIG. 3A is a cross-sectional view of an embodiment of a diagnostic test strip having a test pad in an indentation in the carrier strip.

FIG. 3B is a top view of an embodiment of a diagnostic test strip having a test pad in an indentation in the carrier strip.

FIG. 3C is a perspective view of an embodiment of a diagnostic test strip having a test pad in an indentation in the carrier strip.

FIG. 4A is a cross-sectional view of an embodiment of a diagnostic test strip having two test pads in indentations in a carrier strip.

FIG. 4B is a top view of an embodiment of a diagnostic test strip having two test pads in indentations in a carrier strip.

FIG. 4C is a perspective view of an embodiment of a diagnostic test strip having two test pads in indentations in a carrier strip.

FIG. 5A is a top view of an embodiment of a diagnostic test strip having two test pads on opposite sides of a carrier strip.

FIGS. 5B and 5C are cross-sectional views of an embodiment of a diagnostic test strip having two test pads on opposite sides of a carrier strip.

FIG. 5D is a perspective view of an embodiment of a diagnostic test strip having two test pads on opposite sides of a carrier strip.

FIG. 6A is a top view of an embodiment of a diagnostic test strip having test pads on multiple sides of a carrier strip.

FIG. 6B is a side view of an embodiment of a diagnostic test strip having test pads on multiple sides of a carrier strip.

FIG. 6C is a cross-sectional view of an embodiment of a diagnostic test strip having test pads on multiple sides of a carrier strip.

FIG. 6D is a perspective view of an embodiment of a diagnostic test strip having test pads on multiple sides of a carrier strip.

FIG. 7A is a top view of an embodiment of a diagnostic test strip having two test pads on opposite sides of a carrier strip.

FIGS. 7B and 7C are cross-sectional views of an embodiment of a diagnostic test strip having two test pads on opposite sides of a carrier strip.

FIG. 7D is a perspective view of an embodiment of a diagnostic test strip having two test pads on opposite sides of a carrier strip.

FIG. 8A is a top view of an embodiment of a diagnostic test strip having test pads on multiple sides of a carrier strip.

FIG. 8B is a side view of an embodiment of a diagnostic test strip having test pads on multiple sides of a carrier strip.

FIG. 8C is a cross-sectional view of an embodiment of a diagnostic test strip having test pads on multiple sides of a carrier strip.

FIG. 8D is a perspective view of an embodiment of a diagnostic test strip having test pads on multiple sides of a carrier strip.

FIG. 9A is a cross-sectional view of an embodiment of a diagnostic test strip having multiple layers.

FIG. 9B is a top view of an embodiment of a diagnostic test strip having multiple layers.

FIG. 9C is a perspective view of an embodiment of a diagnostic test strip having multiple layers.

FIG. 10A is a cross-sectional view of an embodiment of a diagnostic test strip having multiple layers and multiple test pads.

FIG. 10B is a top view of an embodiment of a diagnostic test strip having multiple layers and multiple test pads.

FIG. 10C is a perspective view of an embodiment of a diagnostic test strip having multiple layers and multiple test pads.

FIG. 11A is a cross-sectional view of an embodiment of a diagnostic test strip having multiple layers.

FIG. 11B is a top view of an embodiment of a diagnostic test strip having multiple layers.

FIG. 11C is a perspective view of an embodiment of a diagnostic test strip having multiple layers.

FIG. 12A is a cross-sectional view of an embodiment of a diagnostic test strip having multiple layers and multiple test pads.

FIG. 12B is a top view of an embodiment of a diagnostic test strip having multiple layers and multiple test pads.

FIG. 12C is a perspective view of an embodiment of a diagnostic test strip having multiple layers and multiple test pads.

FIG. 13A is a cross-sectional view of an embodiment of a diagnostic test strip having a test pad secured to the carrier strip by a fixing element.

FIG. 13B is a top view of an embodiment of a diagnostic test strip having a test pad secured to the carrier strip by a fixing element.

FIG. 13C is a perspective view of an embodiment of a diagnostic test strip having a test pad secured to the carrier strip by a fixing element.

FIG. 14A is a cross-sectional view of an embodiment of a diagnostic test strip having a test pad secured to the carrier strip by a fixing element.

FIG. 14B is a top view of an embodiment of a diagnostic test strip having a test pad secured to the carrier strip by a fixing element.

FIG. 14C is a perspective view of an embodiment of a diagnostic test strip having a test pad secured to the carrier strip by a fixing element.

### DETAILED DESCRIPTION

The present application relates to U.S. patent application Ser. No. ______, filed ______ entitled “MECHANICAL ATTACHMENT OF TEST PADS TO A DIAGNOSTIC TEST STRIP”, Attorney Docket Number TTUSA.006A2, U.S. patent application Ser. No. ______, filed ______ entitled “MECHANICAL ATTACHMENT OF TEST PADS TO A DIAGNOSTIC TEST DEVICE”, Attorney Docket Number TTUSA.007A2, U.S. patent application Ser. No. ______, filed ______ entitled “DIAGNOSTIC TEST STRIP WITH SELF-ATTACHING TEST PADS AND METHODS OF USE THEREFORE”, Attorney Docket Number TTUSA.008A2, U.S. patent application Ser. No. ______, filed ______ entitled“DIAGNOSTIC TEST STRIPS WITH FLASH MEMORY DEVICES AND METHODS OF USE THEREFORE”, Attorney Docket Number TTUSA.009A2, U.S. patent application Ser. No. ______, filed ______ entitled “DIAGNOSTIC TEST STRIP FOR ORAL SAMPLES AND METHOD OF USE THEREFORE”, Attorney Docket Number TTUSA.010A2, U.S. patent application Ser. No. ______, filed ______ entitled “DIAGNOSTIC TEST STRIPS HAVING ONE OR MORE TEST PAD LAYERS AND METHOD OF USE THEREFORE, Attorney Docket Number TTUSA.011A2, U.S. patent application Ser. No. ______, filed ______ entitled “SINGLE USE MEDICAL TEST PACKAGING”, Attorney Docket Number TTUSA.012A2, U.S. patent application Ser. No. ______, filed ______ entitled “DIAGNOSTIC TEST STRIPS FOR DETECTION OF PAST OR PRESENT INFECTION OF VARIOUS STRAINS OF HEPATITIS” Attorney Docket Number TTUSA.013A2, and U.S. patent application Ser. No. filed ______ entitled“DIAGNOSTIC TEST STRIPS FOR DETECTION OF PRE-SPECIFIED BLOOD ALCOHOL LEVEL” Attorney Docket Number TTUSA.014A2, all of whom have the inventors Ted Titmus and William Pat Price, all of which are filed herewith this even date, all of the disclosures of which are hereby expressly incorporated by reference in their entirety and are hereby expressly made a portion of this application.

Features of the present disclosure will become more fully apparent from the following description and appended claims, taken in conjunction with the accompanying drawings. It will be understood these drawings depict only certain embodiments in accordance with the disclosure and, therefore, are not to be considered limiting of its scope; the disclosure will be described with additional specificity and detail through use of the accompanying drawings. Descriptions of unnecessary parts or elements may be omitted for clarity and conciseness, and like reference numerals refer to like elements throughout. In the drawings, the size and thickness of layers and regions may be exaggerated for clarity and convenience. An apparatus, system or method according to some of the described embodiments can have several aspects, no single one of which necessarily is solely responsible for the desirable attributes of the apparatus, system or method. After considering this discussion, and particularly after reading the section entitled “Detailed Description” one will understand how illustrated features serve to explain certain principles of the present disclosure.

Some embodiments of the invention provide for a diagnostic test strip having an active reference zone containing a carrier strip and one or more test pads having one or more transparent membranes that contain test reagents and/or signaling reagents that detect analytes. Described in more detail below, analytes may be reference analytes, or they may be target analytes. The one or more test pads are optionally located on any side of the carrier strip, including being located on the opposite and/or same side of the carrier strip.

Other embodiments provide for a method of detecting one or more analytes in a patient sample by contacting one or more test pads of an embodiment of a diagnostic test strip with a patient sample and reading the results from the embodiment. Moreover, embodiments may be directly contacted with a patient's sample or the source of the sample. These methods include contacting the test strip with one or more signaling reagents so that the one or more reagents contact the one or more test pads.

Any method's results may be read visually by an embodiment's user, if the application so desires, and/or any method's results may be stored in a memory device for recordation and later access. Alternatively, the results may be read by someone other than the user or the supplier of the sample. In some circumstances, the results of the method will be restricted from the user of the embodiment and/or the supplier of the sample analyzed.

Embodiments of the invention can be used to detect any analyte which has heretofore been assayed using known immunoassay procedures, or known to be detectable by such procedures. Furthermore, it is envisioned that known methods can be modified as needed to afford suitable test reagents and/or signaling reagents that will detect analytes that are similar to analytes that have been previously detected using known procedures.

As disclosed below, various features of the embodiments and methods of using the embodiments enable both trained and untrained personnel to reliably detect the presence, absence, and/or concentration of one or more analytes in a sample. Indeed, features of the embodiments and methods for their use allow for the detection of extremely small quantities of one or more particular analytes while avoiding false positives and false negatives. Furthermore, features of the embodiments and methods for their use allow for accurate and trustworthy attainment and/or storage of information related to the tested sample. Optionally, embodiments may both produce a signal that communicates information to the user and/or store information related to the test sample in one or more memory devices. Consequently, the invention is ideal for use in both prescription and over-the-counter assay test kits which will enable a consumer to self diagnose themselves and others, or test food and/or water prior to consumption.

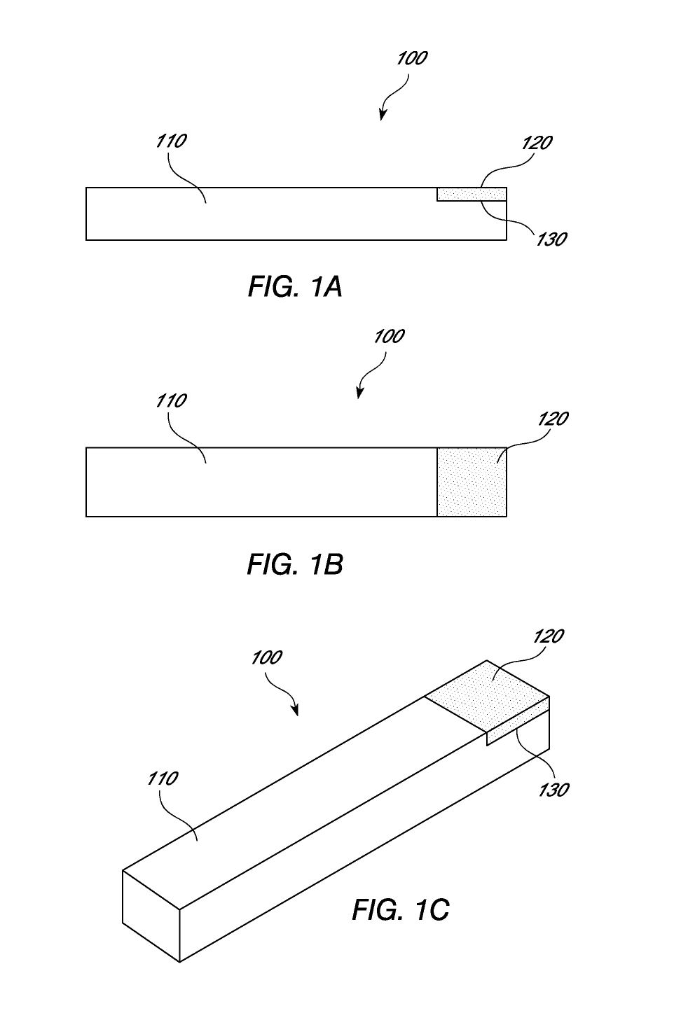

Referring to the drawings, FIGS. 1A, 1B, and 1C illustrate schematically a top view of an embodiment of a diagnostic test strip, 100. FIG. 1A shows a cross-sectional view of the diagnostic test strip, 100, FIG. 1B shows a top view of the diagnostic test strip, 100, and FIG. 1C shows a perspective view of the diagnostic test strip, 100. In this embodiment, the diagnostic test strip, 100, includes a carrier strip, 110, and a test pad, 120. In this embodiment, the carrier strip, 110, includes an indentation, 130, which contains the test pad, 120. In this embodiment, the test pad, 120, has a top surface which substantially aligns with the top surface of the carrier strip, 110. In addition, the test pad, 120 has a bottom surface which is disposed on the carrier strip, 110, in the indentation, 130. Other arrangements may be practiced.

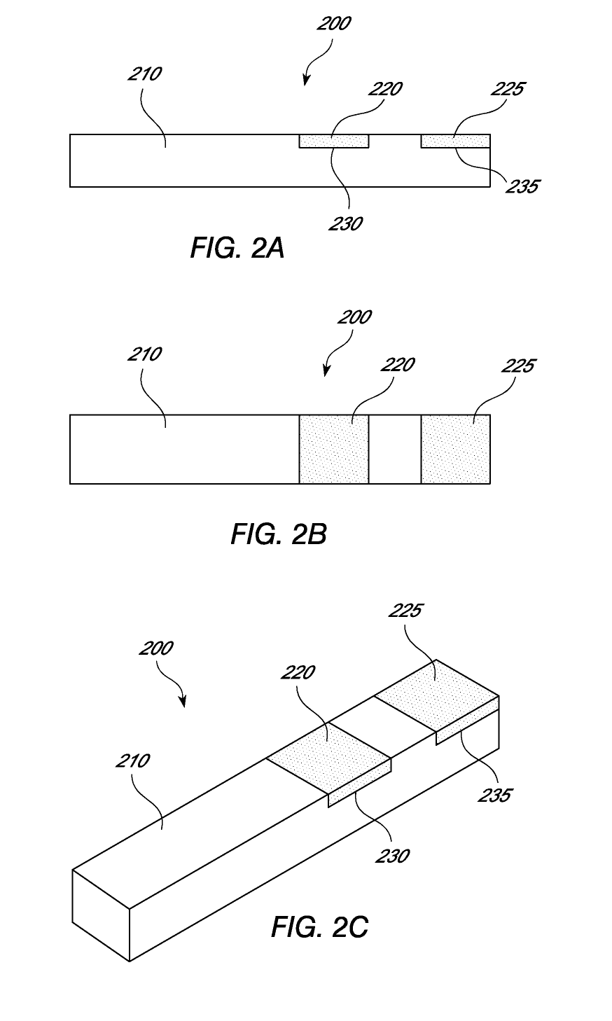

FIGS. 2A, 2B, 2C illustrate an alternative embodiment of a diagnostic test strip, 200. FIG. 2A shows a cross-sectional view of the diagnostic test strip, 200, FIG. 2B shows a top view of the diagnostic test strip, 200, and FIG. 2C shows a perspective view of the diagnostic test strip, 200. In this embodiment, the diagnostic test strip, 200, includes a carrier strip, 210, and test pads, 220 and 225. In this embodiment, the carrier strip, 210, includes indentations, 230 and 235, which respectively contain the test pads, 220 and 225. In this embodiment, the test pads, 220 and 225, each have a top surface which substantially aligns with the top surface of the carrier strip, 210. In addition, the test pads, 220 and 225 each have a bottom surface which is disposed on the carrier strip, 210, in the indentations, 230 and 235, respectively. Other arrangements may be practiced.

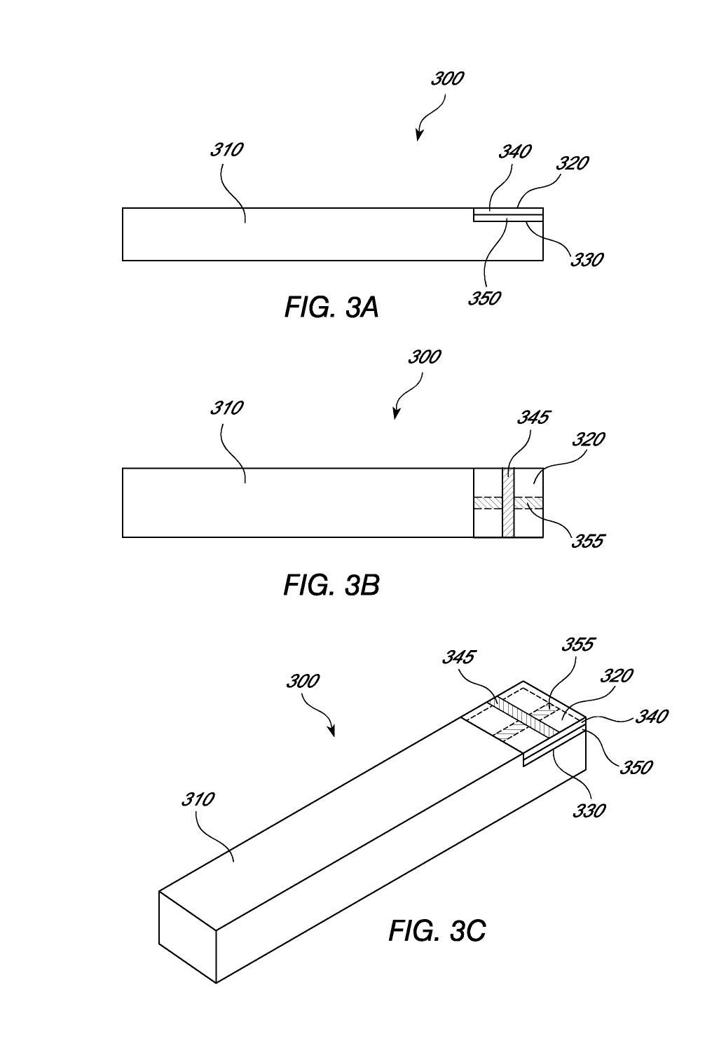

Referring to the drawings, FIGS. 3A, 3B, and 3C illustrate schematically a top view of an embodiment of a diagnostic test strip, 300. FIG. 3A shows a cross-sectional view of the diagnostic test strip, 300, FIG. 3B shows a top view of the diagnostic test strip, 300, and FIG. 3C shows a perspective view of the diagnostic test strip, 300. In this embodiment, the diagnostic test strip, 300, includes a carrier strip, 310, and a test pad, 320. In this embodiment, the carrier strip, 310, includes an indentation, 330, which contains the test pad, 320. In this embodiment, the test pad, 320, has a top surface which substantially aligns with the top surface of the carrier strip, 310. In addition, the test pad, 320 has a bottom surface which is disposed on the carrier strip, 310, in the indentation, 330. Other arrangements may be practiced. Test pad 320 is illustrated as comprising at least two test pad layers, 340 and 350. Consequently, analyte detection by test pad 320 can result in the production of two or more lines resulting from signals 345 and 355. Test pad layers 340 and 350 are capable of generating signals 345 and 355 upon detection of the same analyte, different analytes, and/or different markers for the same analyte.

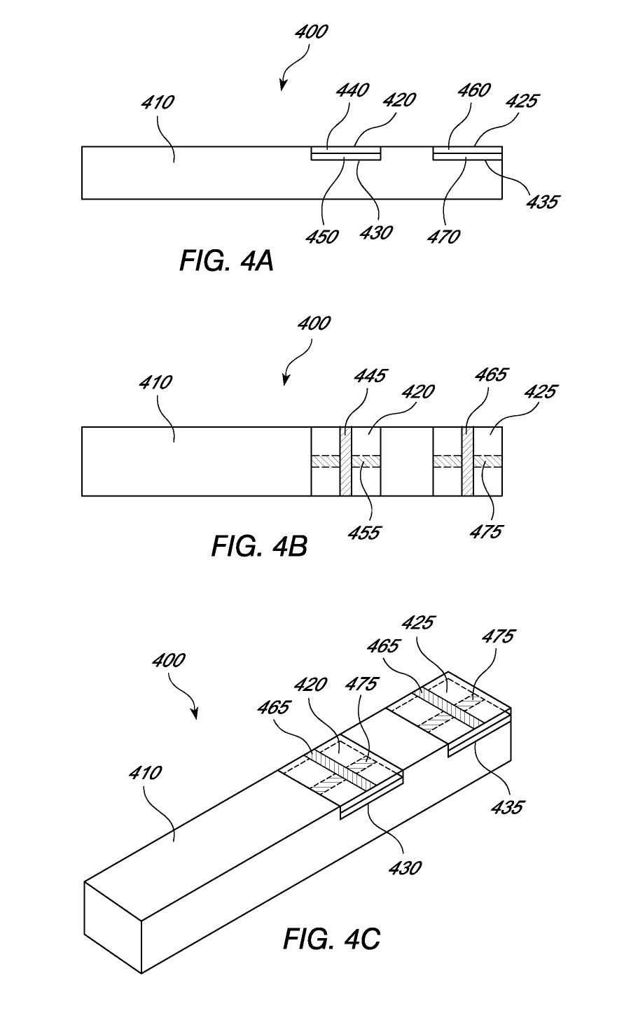

FIGS. 4A, 4B, 4C illustrate an alternative embodiment of a diagnostic test strip, 400. FIG. 4A shows a cross-sectional view of the diagnostic test strip, 400, FIG. 4B shows a top view of the diagnostic test strip, 400, and FIG. 4C shows a perspective view of the diagnostic test strip, 400. In this embodiment, the diagnostic test strip, 400, includes a carrier strip, 410, and test pads, 420 and 425. In this embodiment, the carrier strip, 410, includes indentations, 430 and 435, which respectively contain the test pads, 420 and 425. In this embodiment, the test pads, 420 and 425, each have a top surface which substantially aligns with the top surface of the carrier strip, 410. In addition, the test pads, 420 and 425 each have a bottom surface which is disposed on the carrier strip, 410, in the indentations, 430 and 435, respectively. Other arrangements may be practiced. Test pad 420 is illustrated as comprising at least two test pad layers, 440 and 450. Consequently, analyte detection by test pad 420 can result in the production of two or more lines resulting from signals 445 and 455. Test pad layers 440 and 450 are capable of generating signals 445 and 455 upon detection of the same analyte, different analytes, and/or different markers for the same analyte. Test pad 425 is illustrated as comprising at least two test pad layers, 460 and 470. Consequently, analyte detection by test pad 425 can result in the production of two or more lines resulting from signals 465 and 475. Test pad layers 460 and 470 are capable of generating signals 465 and 475 upon detection of the same analyte, different analytes, and/or different markers for the same analyte.

In alternative embodiments, the test pads may contain one or more test reagents. In addition, each of the indentations may extend substantially across the width of the test strip. In some embodiments, one indentation is on top of the test strip and one indentation is on the bottom of the test strip. Furthermore, the test pads in each of two indentations on a single diagnostic test strip may contain a different test reagent. In some embodiments, a single test pad contains more than one test reagent. In addition, the top surface of the one or more test pads may be below or above the surface of the test strip. In some embodiments, at least one indentation is sealed with a removable cover. Furthermore, at least one indentation may be at one end of the test strip. In some embodiments, at least one indentation contains a plurality of test pads. Each of the plurality of test pads may contain at least one different test reagent. The test strip or the test pad may be substantially porous or may be substantially non-porous.

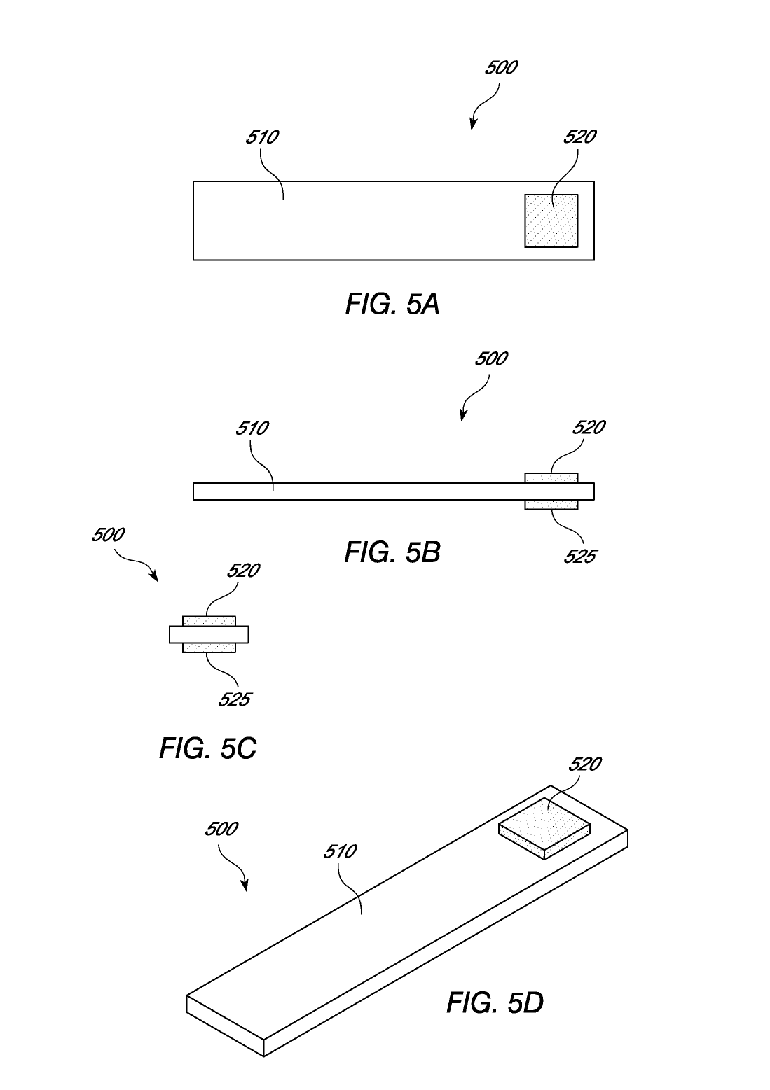

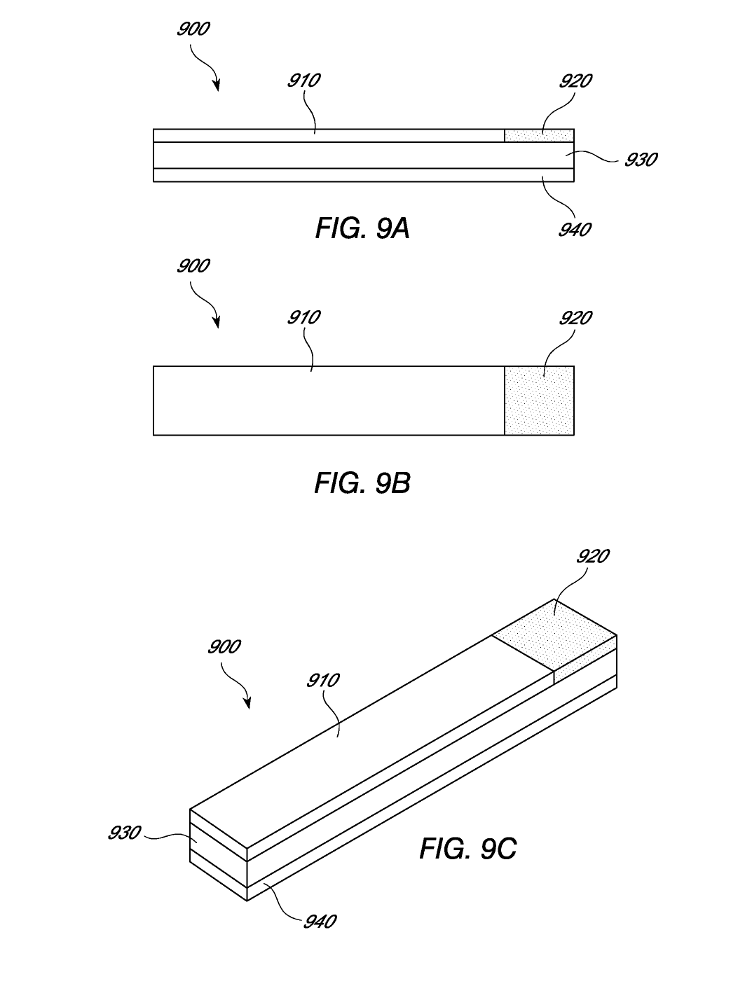

FIGS. 5A, 5B, 5C, and 9D illustrate an alternative embodiment of a diagnostic test strip, 500. FIG. 5A shows a top view of the diagnostic test strip, 500, FIG. 5B shows a cross-sectional view of the diagnostic test strip, 500, FIG. 5C shows a second cross-sectional view of the diagnostic test strip, 500, and FIG. 5D shows a perspective view of the diagnostic test strip, 500. In this embodiment, the diagnostic test strip, 500, includes a carrier strip, 510, and test pads, 520 and 525. In this embodiment, the test pads 520 and 525 are on opposing sides of the carrier strip, 510 and extend above the surface of the carrier strip, 510. Other arrangements may be practiced.

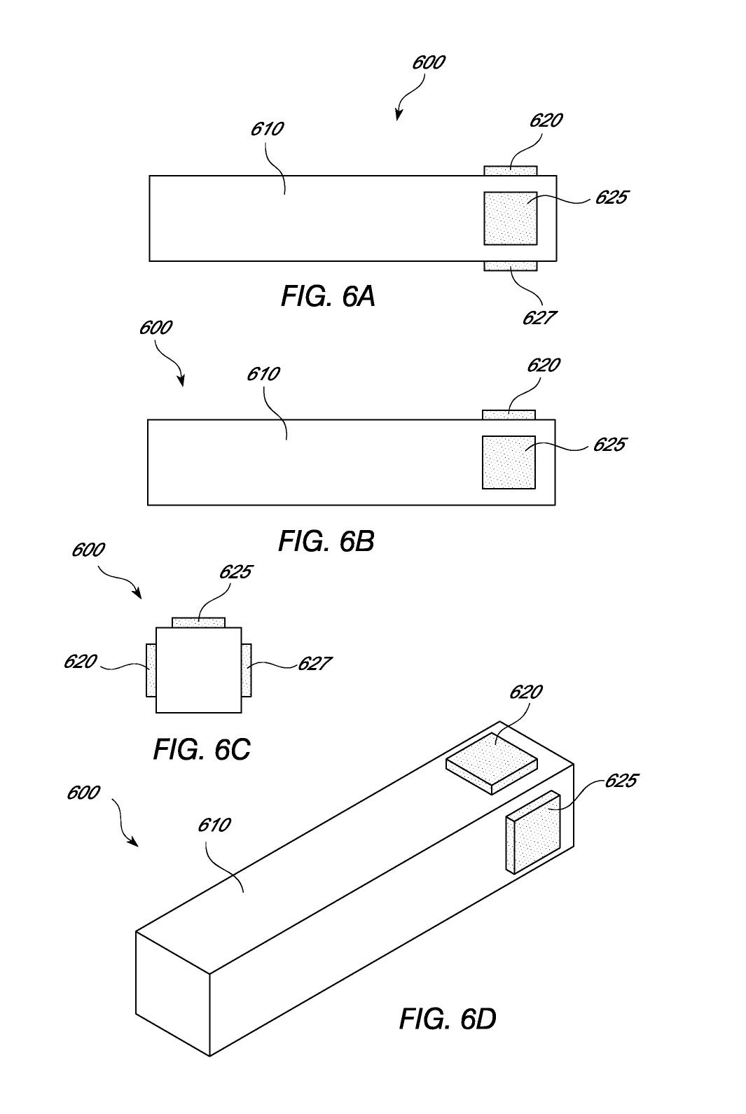

FIGS. 6A, 6B, 6C, and 6D illustrate an alternative embodiment of a diagnostic test strip, 600. FIG. 6A shows a top view of the diagnostic test strip, 600, FIG. 6B shows a side view of the diagnostic test strip, 600, FIG. 6C shows a cross-sectional view of the diagnostic test strip, 600, and FIG. 6D shows a perspective view of the diagnostic test strip, 600. In this embodiment, the diagnostic test strip, 600, includes a carrier strip, 610, and test pads, 620, 625, and 627. In this embodiment, the test pads 620 and 627 are on opposing sides of the carrier strip, 610 and each of the test pads 620, 625, and 627 extend above the surface of the carrier strip, 610. Other arrangements may be practiced.

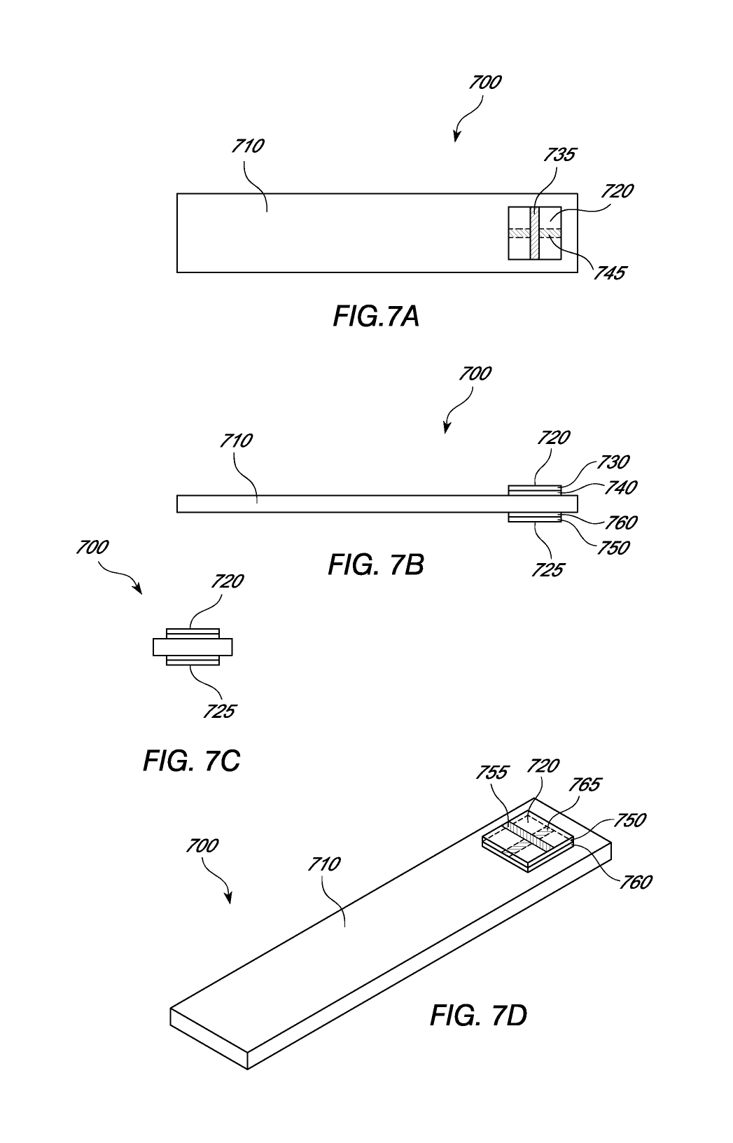

FIGS. 7A, 7B, 7C, and 7D illustrate an alternative embodiment of a diagnostic test strip, 700. FIG. 7A shows a top view of the diagnostic test strip, 700, FIG. 7B shows a cross-sectional view of the diagnostic test strip, 700, FIG. 7C shows a second cross-sectional view of the diagnostic test strip, 700, and FIG. 7D shows a perspective view of the diagnostic test strip, 700. In this embodiment, the diagnostic test strip, 700, includes a carrier strip, 710, and test pads, 9720 and 725. In this embodiment, the test pads 720 and 725 are on opposing sides of the carrier strip, 710 and extend above the surface of the carrier strip, 710. Other arrangements may be practiced. Test pad 720 is illustrated as comprising at least two test pad layers, 730 and 740. Consequently, analyte detection by test pad 720 can result in the production of two or more lines resulting from signals 735 and 745. Test pad layers 730 and 740 are capable of generating signals 735 and 745 upon detection of the same analyte, different analytes, and/or different markers for the same analyte. Test pad 725 is illustrated as comprising at least two test pad layers, 750 and 760. Consequently, analyte detection by test pad 425 can result in the production of two or more lines resulting from signals 755 and 765. Test pad layers 750 and 760 are capable of generating signals 755 and 765 upon detection of the same analyte, different analytes, and/or different markers for the same analyte.

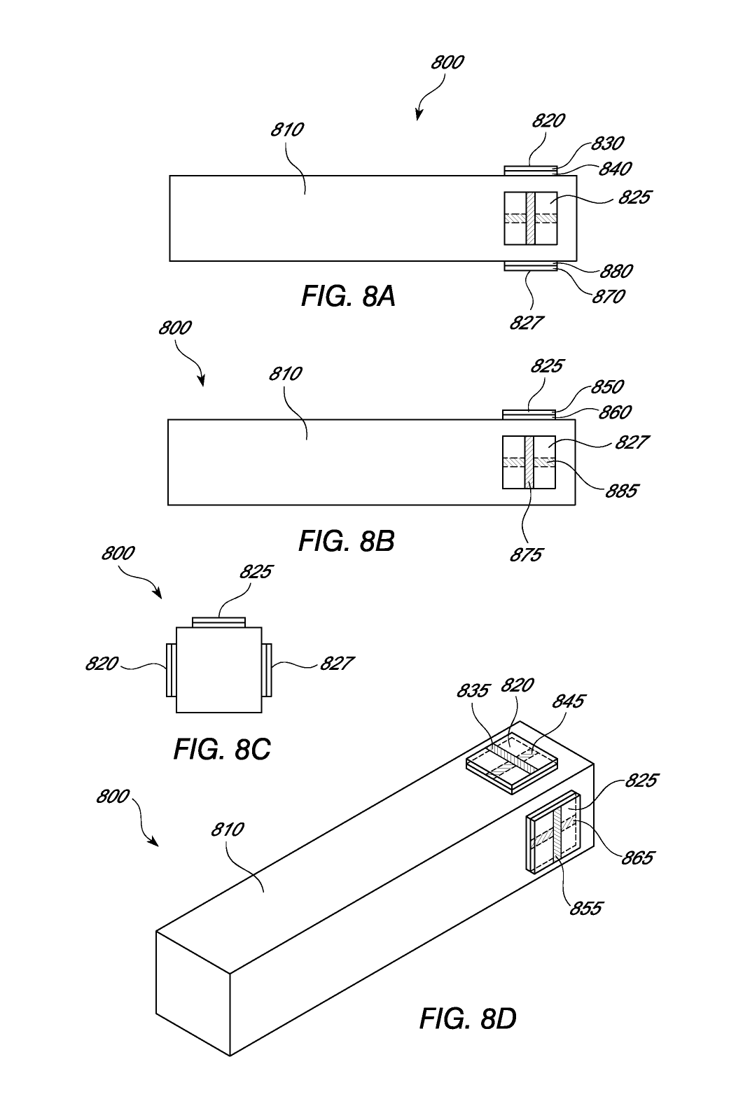

FIGS. 8A, 8B, 8C, and 8D illustrate an alternative embodiment of a diagnostic test strip, 800. FIG. 8A shows a top view of the diagnostic test strip, 800, FIG. 8B shows a side view of the diagnostic test strip, 800, FIG. 8C shows a cross-sectional view of the diagnostic test strip, 800, and FIG. 8D shows a perspective view of the diagnostic test strip, 800. In this embodiment, the diagnostic test strip, 800, includes a carrier strip, 810, and test pads, 820, 825, and 827. In this embodiment, the test pads 820 and 827 are on opposing sides of the carrier strip, 810 and each of the test pads 820, 825, and 827 extend above the surface of the carrier strip, 810. Other arrangements may be practiced. Test pad 820 is illustrated as comprising at least two test pad layers, 830 and 840. Consequently, analyte detection by test pad 820 can result in the production of two or more lines resulting from signals 835 and 845. Test pad layers 830 and 840 are capable of generating signals 835 and 845 upon detection of the same analyte, different analytes, and/or different markers for the same analyte. Test pad 825 is illustrated as comprising at least two test pad layers, 850 and 860. Consequently, analyte detection by test pad 825 can result in the production of two or more lines resulting from signals 855 and 865. Test pad layers 850 and 860 are capable of generating signals 855 and 865 upon detection of the same analyte, different analytes, and/or different markers for the same analyte. Test pad 827 is illustrated as comprising at least two test pad layers, 870 and 880. Consequently, analyte detection by test pad 827 can result in the production of two or more lines resulting from signals 875 and 885. Test pad layers 870 and 880 are capable of generating signals 875 and 885 upon detection of the same analyte, different analytes, and/or different markers for the same analyte.

In alternative embodiments, at least one test pad may contain a test reagent. Each of at the test pads may contain one or more test reagents and the test reagents in each pad may be different. The test reagents may each test for a different marker on the same analyte. In some embodiments, at least one test pad contains a signaling reagent. Furthermore, the carrier strip or at least one test pad may be substantially non-porous or may be substantially porous. In some embodiments, one or more additional test pads are disposed on adjacent sides of the carrier strip. In addition, one or more test pads may extend for substantially the entire width of the side of the strip to which they are disposed.

FIGS. 9A, 9B, 9C illustrate an alternative embodiment of a diagnostic test strip, 900. FIG. 9A shows a cross-sectional view of the diagnostic test strip, 900, FIG. 9B shows a top view of the diagnostic test strip, 900, and FIG. 9C shows a perspective view of the diagnostic test strip, 900. In this embodiment, the diagnostic test strip, 900, includes a carrier strip, 910, a test pad, 920, an intermediate layer, 930, and a bottom laminate layer, 940. In this embodiment, the carrier strip, 910, and the test pad, 920, are on top of the intermediate layer, 930. Accordingly, the carrier strip, 910 is shorter than the intermediate layer, 930, because the test pad, 920 interrupts the pad carrier. In this embodiment, the intermediate layer, 930 is on the top of the bottom laminate layer, 940. Other arrangements may be practiced.

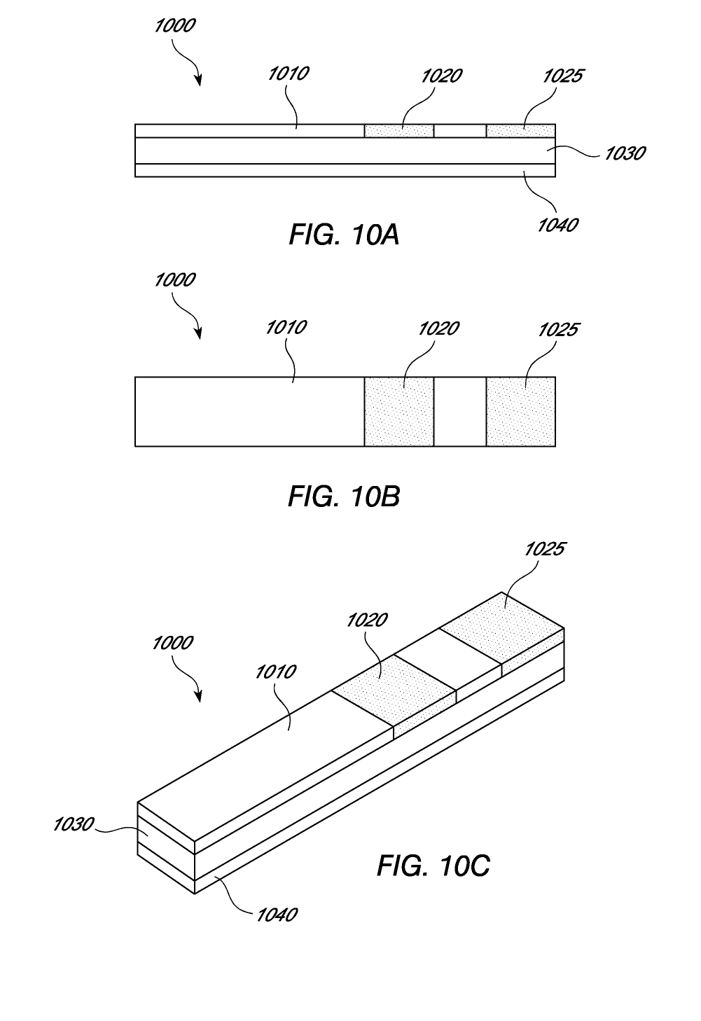

FIGS. 10A, 10B, 10C illustrate an alternative embodiment of a diagnostic test strip, 1000. FIG. 10A shows a cross-sectional view of the diagnostic test strip, 1000, FIG. 10B shows a top view of the diagnostic test strip, 1000, and FIG. 10C shows a perspective view of the diagnostic test strip, 1000. In this embodiment, the diagnostic test strip, 1000, includes a carrier strip, 1010, test pads, 1020 and 1025, an intermediate layer, 1030, and a bottom laminate layer, 1040. In this embodiment, the carrier strip, 1010, and the test pads, 1020 and 1025, are on top of the intermediate layer, 1030. Accordingly, the carrier strip, 1010 is shorter than the intermediate layer, 1030. In this embodiment, the test pads, 1020 and 1025, are interspersed and interrupt the pad carrier. In this embodiment, the intermediate layer, 1030 is on the top of the bottom laminate layer, 1040. Other arrangements may be practiced.

In alternative embodiments, the top of the test pad carrier and the one or more test pads may be substantially flush. In addition, the one or more test pads may extend above the top of the test pad carrier. In some embodiments, there is only one test pad. In addition, the one test pad may be at one end of the test strip. In some embodiments, there are at least two test pads. In addition, each test pad may contain a different test reagent. Furthermore, two test pads may be separated by a length of test carrier pad. In some embodiments, there are more than one test pad and one test pad does not contain a test reagent. In some embodiments, at least one signal generating reagent is present on at least one test pad. The at least one signal generating reagent may be on the test pad containing at least one test reagent. In some embodiments, there are more than one test pads and each test pad detects a different marker on the same analyte.

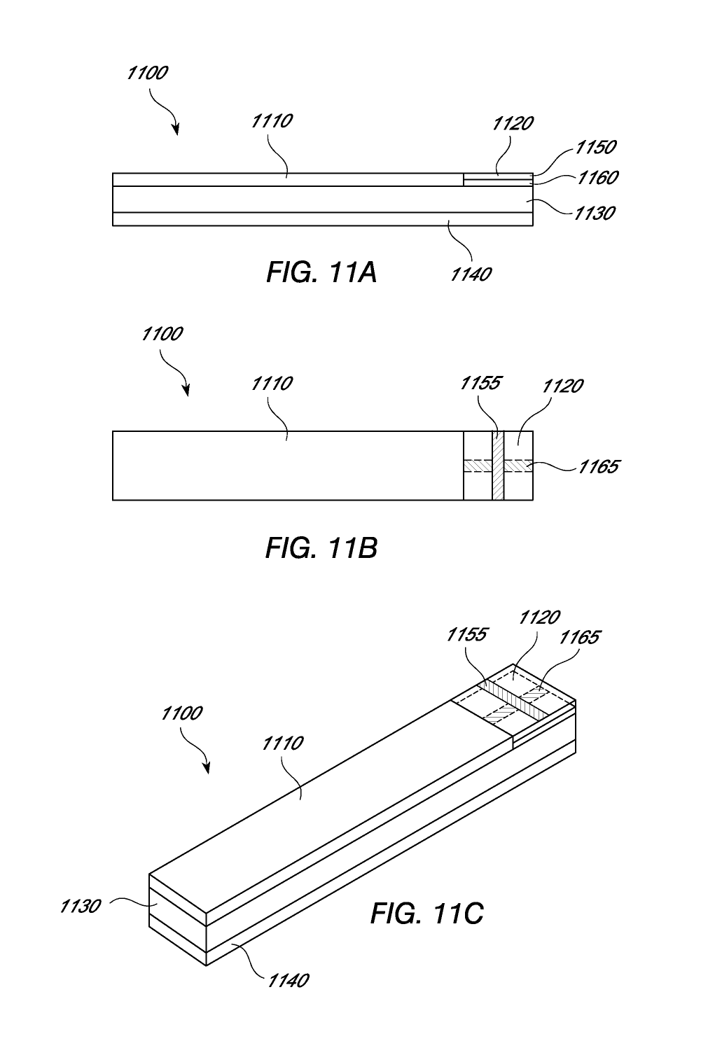

FIGS. 11A, 11B, 11C illustrate an alternative embodiment of a diagnostic test strip, 1100. FIG. 11A shows a cross-sectional view of the diagnostic test strip, 1100, FIG. 11B shows a top view of the diagnostic test strip, 1100, and FIG. 11C shows a perspective view of the diagnostic test strip, 1100. In this embodiment, the diagnostic test strip, 1100, includes a carrier strip, 1110, a test pad, 1120, an intermediate layer, 1130, and a bottom laminate layer, 1140. In this embodiment, the carrier strip, 1110, and the test pad, 1120, are on top of the intermediate layer, 1130. Accordingly, the carrier strip, 1110 is shorter than the intermediate layer, 1130, because the test pad, 1220 interrupts the pad carrier. In this embodiment, the intermediate layer, 1130 is on the top of the bottom laminate layer, 1140. Other arrangements may be practiced. Test pad 1120 is illustrated as comprising at least two test pad layers, 1150 and 1160. Consequently, analyte detection by test pad 1120 can result in the production of two or more lines resulting from signals 1155 and 1165. Test pad layers 1150 and 1160 are capable of generating signals 1155 and 1165 upon detection of the same analyte, different analytes, and/or different markers for the same analyte.

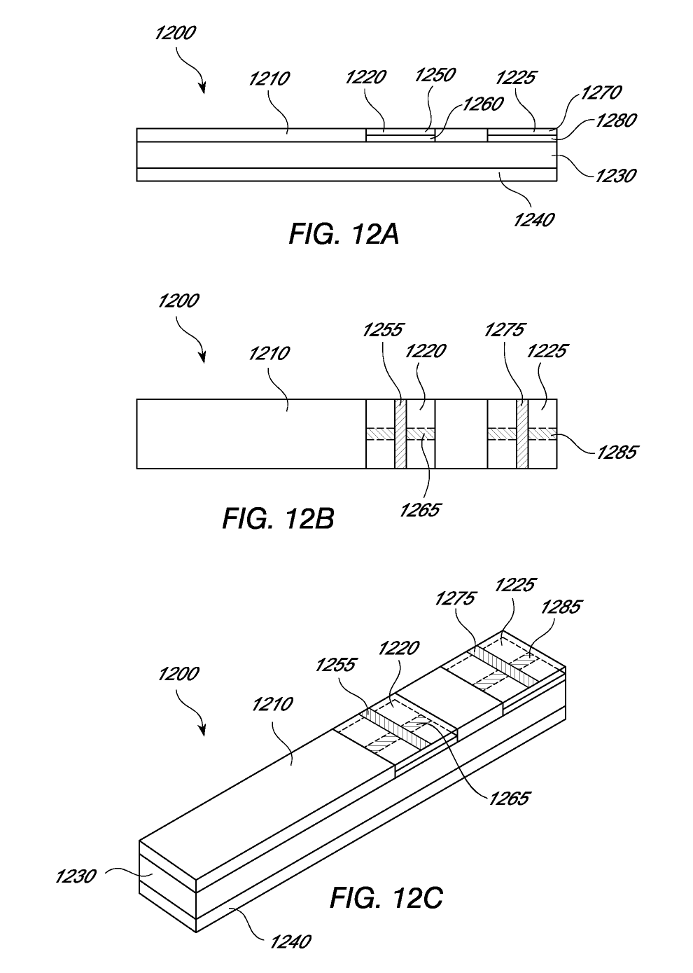

FIGS. 12A, 12B, 12C illustrate an alternative embodiment of a diagnostic test strip, 1200. FIG. 12A shows a cross-sectional view of the diagnostic test strip, 1200, FIG. 12B shows a top view of the diagnostic test strip, 1200, and FIG. 12C shows a perspective view of the diagnostic test strip, 1200. In this embodiment, the diagnostic test strip, 1200, includes a carrier strip, 1210, test pads, 1220 and 1225, an intermediate layer, 1230, and a bottom laminate layer, 1240. In this embodiment, the carrier strip, 1210, and the test pads, 1220 and 1225, are on top of the intermediate layer, 1230. Accordingly, the carrier strip, 1210 is shorter than the intermediate layer, 1230. In this embodiment, the test pads, 1220 and 1225, are interspersed and interrupt the pad carrier. In this embodiment, the intermediate layer, 1230 is on the top of the bottom laminate layer, 1240. Other arrangements may be practiced. Test pad 1220 is illustrated as comprising at least two test pad layers, 1250 and 1260. Consequently, analyte detection by test pad 1220 can result in the production of two or more lines resulting from signals 1255 and 1265. Test pad layers 1250 and 1260 are capable of generating signals 1255 and 1265 upon detection of the same analyte, different analytes, and/or different markers for the same analyte. Test pad 1225 is illustrated as comprising at least two test pad layers, 1270 and 1280. Consequently, analyte detection by test pad 1225 can result in the production of two or more lines resulting from signals 1275 and 1285. Test pad layers 1270 and 1280 are capable of generating signals 1275 and 1285 upon detection of the same analyte, different analytes, and/or different markers for the same analyte.

In alternative embodiments, the top of the test pad carrier and the one or more test pads may be substantially flush. In addition, the one or more test pads may extend above the top of the test pad carrier. In some embodiments, there is only one test pad. In addition, the one test pad may be at one end of the test strip. In some embodiments, there are at least two test pads. In addition, each test pad may contain a different test reagent. Furthermore, two test pads may be separated by a length of test carrier pad. In some embodiments, there are more than one test pad and one test pad does not contain a test reagent. In some embodiments, at least one signal generating reagent is present on at least one test pad. The at least one signal generating reagent may be on the test pad containing at least one test reagent. In some embodiments, there are more than one test pads and each test pad detects a different marker on the same analyte.

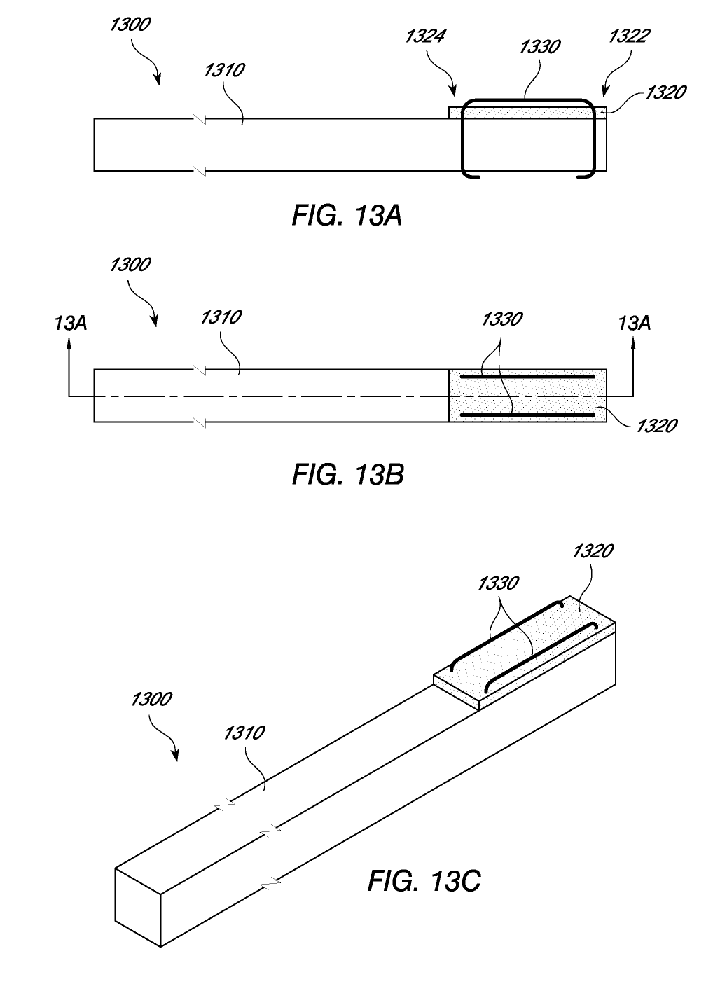

FIGS. 13A, 13B, and 13C illustrate an alternative embodiment of a diagnostic test strip, 1300. FIG. 13A shows a cross-sectional view of the diagnostic test strip, 1300, along line 13A to 13A. FIG. 13B shows a top view of the diagnostic test strip, 1300, and FIG. 13C shows a perspective view of the diagnostic test strip, 1300. In this embodiment, the diagnostic test strip, 1300, includes a carrier strip, 1310, and a test pad 1320. In addition, diagnostic test strip, 1300, has a mechanical fixing element, 1330. As shown, the mechanical fixing elements, 1330, mechanically fix the test pad, 1320, to the carrier strip, 1310. In this embodiment, the mechanical fixing elements, 1330, extend along the top the test pad, 1320, and protrude through leading and trailing portions, 1322 and 1324, of the test pad, 1320. The mechanical fixing elements, 1330, also protrude through the carrier strip, 1310, and extend along the back of the carrier strip, 1310. The mechanical fixing element, 1330, holds the test pad, 1320, to the carrier strip, 1310. Other arrangements may be practiced.

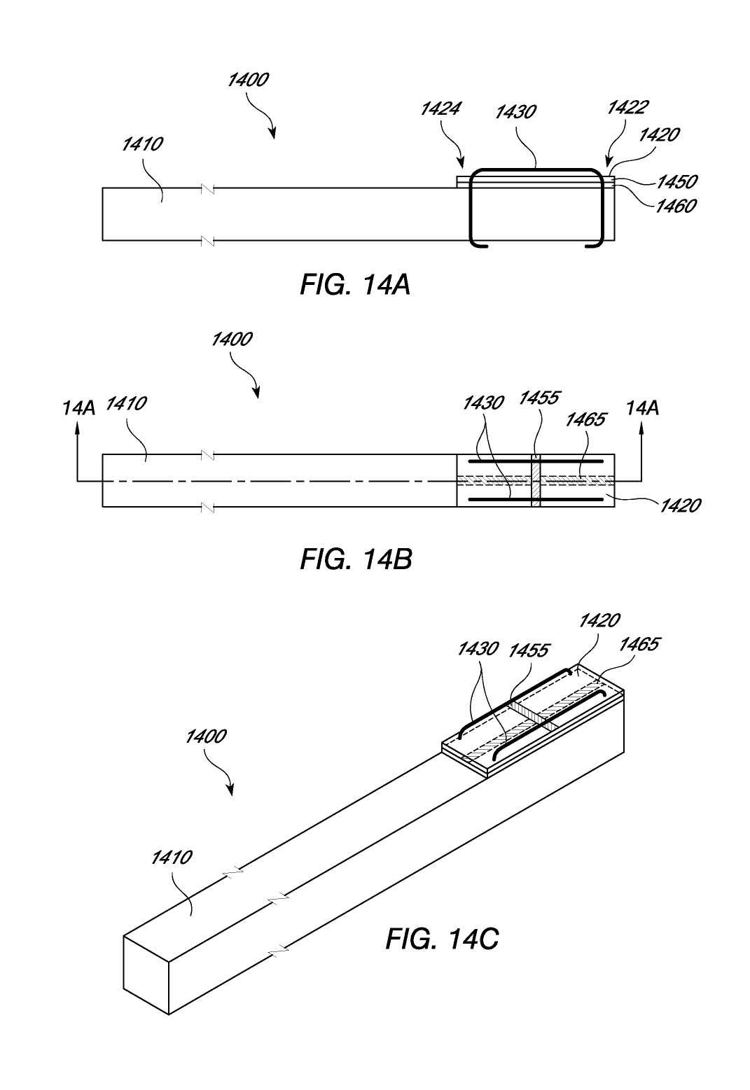

FIGS. 14A, 14B, and 14C illustrate an alternative embodiment of a diagnostic test strip, 1400. FIG. 14A shows a cross-sectional view of the diagnostic test strip, 1400, along line 14A to 14A. FIG. 14B shows a top view of the diagnostic test strip, 1400, and FIG. 14C shows a perspective view of the diagnostic test strip, 1400. In this embodiment, the diagnostic test strip, 1400, includes a carrier strip, 1410, and a test pad 1420. In addition, diagnostic test strip, 1400, has a mechanical fixing element, 1430. As shown, the mechanical fixing elements, 1430, mechanically fix the test pad, 1420, to the carrier strip, 1410. In this embodiment, the mechanical fixing elements, 1430, extend along the top the test pad, 1420, and protrude through leading and trailing portions, 1422 and 1424, of the test pad, 1420. The mechanical fixing elements, 1430, also protrude through the carrier strip, 1410, and extend along the back of the carrier strip, 1410. The mechanical fixing element, 1430, holds the test pad, 1420, to the carrier strip, 1410. Other arrangements may be practiced. Test pad 1420 is illustrated as comprising at least two test pad layers, 1450 and 1460. Consequently, analyte detection by test pad 1420 can result in the production of two or more lines resulting from signals 1455 and 1465. Test pad layers 1450 and 1460 are capable of generating signals 1455 and 1465 upon detection of the same analyte, different analytes, and/or different markers for the same analyte.

In alternative embodiments, the test pad may be fixed to the test strip with a staple. In some embodiments, a first staple bridges the trailing portion of the test pad and a second staple bridges the leading portion of the test pad. In some embodiments, only one staple bridges the trailing portion and only one staple bridges the leading portion of the test pad. A third staple may bridge one side of the test pad and a fourth staple may bridge a second side of the test pad. In some embodiments, there are two or more test pads and at least one is attached to the top of the test strip and at least one is attached to the bottom of the test strip. In some embodiments, there are two or more test pads and at least one is attached to the top of the test strip and at least one other is attached to a side of the test strip. Each of the test pads may contain a test reagent, and at least one of the test pads may further contain a signaling reagent. In some embodiments, each of the test pads contains a different test reagent. Each of the test pads may have a different test reagent and each of the reagents may detect a different marker on the same analyte. In some embodiments, a single test pad is mechanically fixed to the top of the carrier strip. Alternatively, two or more test pads may be mechanically fixed to the top of the carrier strip. In some embodiments, three test pads are mechanically fixed to the top of the carrier strip. In some embodiments, at least one test pad is mechanically fixed to the top of the carrier strip and at least one test pad is attached to the bottom of the carrier strip. One test pad may be mechanically attached to the top of the carrier strip and one may be mechanically attached to a side of the carrier strip. The fixing element may be, for example, metal, paper, or plastic. In some embodiments, the test strip has indentations in the top of the strip and the test pads are fixed to the strip in the indentations. The indentations may contain the entire depth of the test pad such that the top of the test pad is flush with the top of the test strip. The indentations may be such that the top of the test pads are below the top of the test strip. In some embodiments, the test pads extend substantially from side to side on the top of the carrier strip. Test strip or the test pads may be substantially porous or substantially non-porous.

### Carrier Strip

The carrier strip provides structural support for the one or more test pads and the one or more boundary projections. As a structural support, many materials suitable for use in preparing the carrier strip are known in the art. Such materials include but are not limited to plastics including polyethylene terephthalate, high-density polyethylene, polypropylene, cellulose, Bakelite, polystyrene, high impact polystyrene, acrylonitrile butadiene styrene, polyester, polyurethanes, polycarbonates, polycarbonate/acrylonitrile butadiene styrene, polymethyl methacrylate, polytetrafluoroethylene, polyetherimide, phenol formaldehydes, urea-formaldehyde, melamine formaldehyde, polylactic acid, plastarch-material, polyvinylchloride, nylon, and other polyamides, metals, alloys, ceramics, glass, wood, cardboard, paper, natural rubber, synthetic rubber, and other suitable polymers. Optionally, the carrier strip may be porous or non-porous. Optionally, the carrier strip may facilitate the transmission of information from the one or more test pads to a memory device. Transmitted information may include, but is not limited to, the presence, absence, and/or concentration of one or more analytes of interest. The carrier strip may facilitate the transmission of information from the one or more test pads to the one or more memory devices by any of several methods known in the art. Such methods include, but are not limited to, the transmission of electrical signals which result from changes in the coulometry, amperometry, or potentiometry of the materials comprising the carrier strip. See U.S. Pat. No. 6,743,635 (Neel et al., issued on Jun. 1, 2001) and U.S. Pat. No. 6,946,299 (Neel at al., issued on Sep. 20, 2005), which are herein incorporated by reference. Alternatively, the carrier strip may facilitate the transmission of optical signals which result from differences in the reflection, transmission, scattering, absorption, fluorescence, or electrochemiluminescense of the materials comprising the carrier strip and/or the test pads. See U.S. Pat. No. 6,040,195 (Carroll et al., issued on Mar. 21, 2000) and U.S. Pat. No. 6,284,550 (Carroll et al., issued on Sep. 4, 2001) which are herein incorporated by reference.

The carrier strip's size and shape is only limited by the desired application of the embodiment. For example, if the desired application is testing a human patient, the embodiment, and consequently the carrier strip, may be smaller or larger depending upon the size of the human patient. Likewise, if the desired application involves testing an animal patient, the embodiment, and consequently the carrier strip, may be smaller or larger depending upon the size of the animal patient. In some embodiments, the carrier strip is about 1, about 1.25, about 1.5, about 1.75, about 2, about 2.25, about 2.5, about 2.75, about 3, about 3.25, about 3.5, about 3.75, about 4, about 1-2, about 1-3, about 1-4, about 2-3, about 2-4, or about 3-4 inches in length. The carrier strip's shape may optionally be varied depending upon the desired application of the embodiment. Some applications may require substantially narrow, fat, rectangular, circular, oval, square, triangular, or other shapes, including combinations of the indicated shapes. It is envisioned that the shape of embodiments can be tailored to the shape of the environment in which the embodiments will be applied. Moreover, the carrier strip may contain boundary projections that substantially surround one, two, three, and/or four sides of one or more test pads to collect and/or direct sample application to the one or more test pads. Furthermore, it is envisioned that a handle may be optionally attached to a carrier strip or in contact with a carrier strip, either directly or indirectly.

### Test Reagents and Signaling Reagents

Test reagents and signaling reagents suitable for inclusion in embodiments are well known in the art. Such reagents include, but are not limited to, polyclonal antisera and monoclonal antibodies that have specific binding properties and high affinity for virtually any antigenic substance. Literature affords many means of preparing such reagents. See, e.g., Laboratory Techniques in Biochemistry and Molecular Biology, Tijssen, Vol. 15, Practice and Theory of Enzyme Immunoassays, chapter 13, The immobilization of Immunoreactants on Solid Phases, pp. 297-328, and the references cited therein which are herein incorporated by reference. Additional assay protocols, reagents, and analytes useful in the practice of the invention are known per se. See, e.g., U.S. Pat. No. 4,313,734 (Leuvering, issued on Feb. 2, 1982), columns 4-18, and U.S. Pat. No. 4,366,241(Tom et al., issued on Dec. 28, 1982), columns 5-40 which are herein incorporated by reference.

Metal sols, including but not limited to gold sol, and other types of colored particles, including but not limited to, organic dye sols and colored latex particles, that are useful as marker substances in immunoassay procedures are also known per se and suitable for use as test reagents and/or signaling reagents. See, for example, U.S. Pat. No. 4,313,734 (Leuvering, issued on Feb. 2, 1982), the disclosure of which is incorporated herein by reference. For details and engineering principles involved in the synthesis of colored particle conjugates see Horisberger, Evaluation of Colloidal Gold as a Cytochromic Marker for Transmission and Scanning Electron Microscopy, Biol. Cellulaire, 36, 253-258 (1979); Leuvering et al, Sol Particle Immunoassay, J. Immunoassay 1 (1), 77-91 (1980), and Frens, Controlled Nucleation for the Regulation of the Particle Size in Monodisperse Gold Suspensions, Nature, Physical Science, 241, pp. 20-22 (1973) which are herein incorporated by reference.

Test reagents for inclusion in the embodiments may signal directly, such as with an electrical or optical signal (visible either to the naked eye, or with an optical filter or upon applied stimulation to promote fluorescence or phosphorescence). Test reagents may also signal indirectly such as with enzymes, e.g. alkaline phosphatase and/or horseradish peroxidase, in combination with signaling reagents in the form of enzymatic substrates that will generate a signal upon interaction with the enzyme. In some embodiments, the signaling reagent and/or test reagent is incorporated into the test pad. In other embodiments, the signaling reagent and/or test reagent is added to the test sample before application to the test pad. In additional embodiments, the signaling reagent and/or test reagent is added to the test pad after introduction of the test sample.

Alcohol sensitive test reagents and methods are well known in the art. See, e.g. U.S. Pat. No. 5,563,073 (Titmas, issued on Oct. 8, 1996) and Jai Moo Shin et al., Simple Diagnostic Tests to Detect Toxic Alcohol Intoxications, NIH (October 2008), which are hereby incorporated by reference in their entirety. In some embodiments, the test reagent and/or signaling reagent from Alco Screen™ pads, manufactured by Chematics, Inc. located in North Webster, Ind., is incorporated. Optionally, the test reagent and/or signaling reagent from Alco Screen™ pads is incorporated in the one or more test pads, but it may also be applied to the test pad after sample application or it may be applied to the sample before application to the test pad. In some embodiments the test reagent and/or signaling reagent from the alcohol dehydrogenase method (ADH method) is incorporated in the one or more test pads, but it may also be applied to the test pad after sample application or it may be applied to the sample before application to the test pad. In some embodiments the test reagent and/or signaling reagent from the alcohol oxidase method method (ALOx method) is incorporated in the one or more test pads, but it may also be applied to the test pad after sample application or it may be applied to the sample before application to the test pad. In some embodiments the test reagent and/or signaling reagent from the sodium periodate method is incorporated in the one or more test pads, but it may also be applied to the test pad after sample application or it may be applied to the sample before application to the test pad. In some embodiments the test reagent and/or signaling reagent from the potassium permanganate method (PA method) is incorporated in the one or more test pads, but it may also be applied to the test pad after sample application or it may be applied to the sample before application to the test pad.

Test reagents and/or signaling reagents may also detect the storage and handling of embodiments. In some embodiments, test reagents and/or signaling reagents may be sensitive to temperature and if the temperature of the embodiment's environment has exceeded or fallen below a predetermined temperature, optionally for a predetermined period of time, the test reagents and/or signaling reagents may be inactivated. Optionally, the inactivation of the test reagents and/or signaling reagents may result in the transmission of a signal to the one or more memory devices and/or to the user of the embodiment.

In some embodiments, test reagents and/or signaling reagents may be sensitive to moisture, and if the humidity of the embodiment's environment has exceeded or fallen below a predetermined level, optionally for a predetermined period of time, the test reagents and/or signaling reagents may be inactivated. Optionally, the inactivation of the test reagents and/or signaling reagents may result in the transmission of a signal to the one or more memory devices and/or to the user of the embodiment.

Test reagents and/or signaling reagents may also detect whether a sufficient amount of sample has been applied to an embodiment for analysis. For example, when the sample is saliva, a test reagent and/or signaling reagent specific for a salivary enzyme, such as amylase, may detect the salivary enzyme's presence if a sufficient volume of sample has been applied. The detection of a sufficient sample may optionally be signaled to the user in the form of a color or symbol. Using such embodiments, the user would then know if a sufficient quantity of sample was applied to the one or more test pads to afford an accurate analysis.

Embodiments that detect storage and/or sufficient application of sample volume are particularly capable of reducing the occurrence of false negatives. For example, poor storage conditions may inactivate a test reagent in a test pad. Upon application of sample to such a test pad, no signal may result and a user could believe that an analyte is not present—a false negative. Alternatively, test pads having a pre-printed negative signal may suffer a similar occurrence of a false negative if the test reagent is inactivated because an analytes presence in a sample would not convert the pre-printed negative signal into a positive signal. Likewise, an insufficient volume of sample may generate no signal or a negative signal and cause a user to believe that an analyte is not present.

Any enzyme, antibody, dye buffer, chemical, sol, or combinations thereof may be incorporated so long as the enzyme, antibody, dye buffer, chemical, metal sol, or combinations thereof are capable of detecting the presence of one or more analytes in a sample. See, e.g., U.S. Pat. No. 6,383,736 (Titmas, issued on May 7, 2002), U.S. Pat. No. 7,858,756 (Owens et al., issued on Dec. 28, 2010), and U.S. Pat. No. 7,790,400 (Jehanli et al., issued on Sep. 7, 2010) which are hereby incorporated by reference in their entirety.

### Test Pads

The one or more test pads may be prepared from any bibulous, porous, fibrous, or sorbent material capable of rapidly absorbing a sample. Porous plastics material, such as polypropylene, polyethylene, polyvinylidene flouride, ethylene vinylacetate, acrylonitrile and polytetrafluoroethylene can be used. Optionally, the one or more test pads can be pre-treated with a surface-active agent to reduce any inherent hydrophobicity in the one or more test pads and enhance their ability to absorb a sample. Moreover any one of the one or more test pads may be treated with an oxygen-impermeable water soluble substance. Suitable examples of an oxygen-impermeable water soluble substance include, but are not limited to, polyvinyl alcohol, partly saponified polyvinyl acetate which can also contain vinylether and vinylacetal units, polyvinyl pyrrolidone and copolymers thereof with vinyl acetate and vinyl ethers, hydroxy alkyl cellulose, gelatin, polyacrylic acid, gum arabic, polyacryl amide, dextrin, cyclodextrin, copolymers of alkylvinyl ethers and maleic acid anhydride, ring opened polymers of maleic acid anhydride, water-soluble high molecular polymers of ethylene oxide having molecular weights of above 5,000, and/or polyvinyl alcohol in combination with poly(l-vinylimidazole) or a copolymer of 1-vinyl-imidazole. The one or more test pads can also be made from paper or other cellulosic materials, including but not limited to nitrocellulose. Materials that are now used in the nibs of fiber-tipped pens are also suitable for incorporation in the one or more test pads.

Optionally, the one or more test pads may be prepared from non-porous materials. In such circumstances, the test reagents and/or signaling reagents may be coated on the outer surface of the one or more test pads such that contact with a sample containing an analyte will result in the generation of a signal.

Using known methods, test pads may be shaped or extruded in a variety of lengths and cross-sections. Embodiments may possess one or more test pads of various sizes and shapes, and the size and shape of the one or more test pads are only limited by their number, size, and desired application of the embodiment in which they are incorporated within. In some embodiments, the one or more test pads are substantially similar in size and/or shape. In other embodiments, the one or more test pads may differ substantially in size and/or shape. It is readily envisioned that embodiments may possess about one or more test pads, about two or more test pads, about three or more test pads, about four or more test pads, about five or more test pads, about six or more test pads, about seven or more test pads, about eight or more test pads, about nine or more test pads, about ten or more test pads, about 1-4 test pads, about 1-10 test pads about 1-100 test pads, about 2-100 test pads, about 3-100 test pads, about 4-100 test pads, about 5-100 test pads, about 5-75 test pads, about 10-50 test pads, about 15-25 test pads, and individual numbers of test pads therein. The one or more test pads may be made of the same material, or optionally they may be made of different materials or even combinations of different materials. Moreover, the one or more test pads may be recessed into the carrier strip.

In some embodiments, test pads may be prepared from a single layer of material. In other embodiments, test pads may be prepared from multiple layers of material. It is readily envisioned that embodiments may possess about one or more layers, about two or more layers, about three or more layers, about four or more layers, about five or more layers, about six or more layers, about seven or more layers, about eight or more layers, about nine or more layers, about ten or more layers, about 1-4 layers, about 1-5 layers, about 1-6 layers, about 1-7 layers, about 1-8 layers, about 1-9 layers, about 1-10 layers, about 1-100 layers, about 2-100 layers, about 3-100 layers, about 4-100 layers, about 5-100 layers, about 5-75 layers, about 10-50 layers, about 15-25 layers, and individual numbers of layers therein.

The test pad layers may be of the same or different materials. Test reagents and/or signaling reagents may also be impregnated in a single layer of material or in multiple layers of material. The impregnation may take any suitable form, including, but not limited to, a substantially uniform impregnation or impregnation with dots or stripes. Test reagents and/or signaling reagents can be impregnated in various concentrations in one or more of the multiple layers to tailor the sensitivity of the test pads to certain analytes. Such sensitivity could afford information about the concentration of an analyte in the sample. Furthermore, the impregnation may optionally be conducted in a manner that will generate a signal observable by the user upon application of a sufficient quantity of sample, detection of an analyte, or proper/improper storage of the embodiment.

When one or more test pads are comprised of multiple layers of material, one or more layers of material may be impregnated (e.g. pre-printed) with an inert chemical such that a line or “minus sign” is displayed to the user. In some embodiments, the line or “minus sign” could be in the form of a material covering the one or more test pads to give a visual impression of a line or “minus sign” on the one or more test pads. One or more additional layers of the material comprising the one or more test pads could then be impregnated with a test reagent and/or a signaling reagent that upon detecting a sufficient quantity of sample, appropriate storage temperature, and/or the presence of an analyte, the impregnated test reagent and/or signaling reagent will create a perpendicular line such that a “plus sign” will be signaled to the user. In other embodiments, the line or “minus sign” displayed in the one or more test pads could be obscured by color or opaqueness when a test reagent and/or a signaling reagent detects a sufficient quantity of sample, appropriate or inappropriate storage temperature, and/or the presence of an analyte.

The test pad layers may comprise optically transparent membranes. Detection on an analyte may then generate a signal that is opaque, partially transparent, or completely transparent. Moreover, test pad layers may be only partially optically transparent prior to application of a sample. Alternatively, the application of a sample to one or more test pad layers may result in the layers becoming optically transparent, thereby allowing a user to see generated and/or pre-printed signals on test pad layers below the optically transparent layers. Moreover, the individual layers in a test pad may be positioned such that the detection of an analyte in a lower layer of material is obscured by the detection of an analyte in a layer of material positioned above the lower layer.

It is also envisioned that embodiments may have arrangements of test pads and/or arrangements of layers within multiple layered test pads such that the detection of an analyte in the test pads or the layers of a test pad generate a signal, such as a “plus sign” or “minus sign” to the user. Such embodiments may comprise at least two layers of material, each capable of generating a line upon detecting an analyte or a certain concentration of an analyte. Optionally, the lines may intersect to generate a “plus” sign or other signal upon the detection of an analyte in the at least two layers of material. Alternatively, embodiments may comprise at least four layers of material, each capable of generating a line upon detecting an analyte or a certain concentration of an analyte in the at least four layers of material. Optionally, the lines may intersect at one or more points such that a “plus” sign or other symbol is formed. While the aforementioned embodiments have been discussed with reference to “minus” and “plus” signs, it is envisioned that any symbol, including color changes, could be used to convey similar information to a user. Such symbols include, but are not limited to, circles, ovals, squares, triangles, trapezoids, rhombi, plus signs, minus signs, “X” shaped signs, checkmarks, and/or dotted, dashed, or differentially colored version of said symbols. The meaning of any desired symbol or color change could be included in the packaging of an embodiment or imprinted on an embodiment.

The test reagents applied to each layer of material may optionally be the same or different. When different test reagents are applied to different layers of material comprising the one or more test pads, the test pad may be tailored to generate a signal indicating the diagnosis of one or more illnesses, diseases, or injuries. One method for achieving such a diagnosis would be to have the individual layers comprising the test pad generate a signal in response to one or more symptoms of one or more illnesses, diseases, or injuries. For example, if the diagnosis of one or more illnesses, diseases, or injuries required the determination of multiple analytes, then the detection of each analyte could produce a portion of a symbol that is visible to the user. Upon formation of a complete symbol, the embodiment would confirm the presence of a certain illness, disease, or injury. Optionally, information relating to each specific analyte could be transferred to the one or more memory devices.

One can readily appreciate the application of such embodiments of multiple layer test pads when knowledge of a certain concentration is needed. As a non-limiting application, the detection of a person's blood alcohol level may be achieved using such an embodiment. For a test pad comprising at least four test pad layers, if a first test pad layer was sensitive to a blood alcohol level of at least 0.02%, a second test pad layer was sensitive to a blood alcohol level of at least 0.04%, a third test pad layer was sensitive to a blood alcohol level of at least 0.06%, and a fourth test pad layer was sensitive to a blood alcohol level of at least 0.08%, then the application of a sample having a blood alcohol level at least at the sensitive percentages would generate a signal. Assuming that operating a motor vehicle with a blood alcohol level equal to or greater than 0.08% is illegal, then the application of a sample that generates a “plus” sign would indicate that the sample provider should not legally operate a motor vehicle. One will readily appreciate that this described example is capable of extension to any number of test pads having any number of layers, such that the detection of an analyte in each layer generates a signal indicative of concentration.

As another non-limiting example, test reagents and/or signaling reagents that are sensitive to markers specific for hepatitis and/or liver damage may be applied to test pads and/or layers within test pads. Consequently, the detection of markers specific for hepatitis and/or liver damage in each test pad and/or layers within test pads would generate a signal. An individual test pad may optionally be sensitive to a single marker for hepatitis and/or liver damage. Alternatively, a single test pad may be sensitive to multiple markers for hepatitis and/or liver damage. In such an embodiment, the detection of one or more markers for hepatitis and/or liver damage may produce a certain signal, e.g. color, indicative of the number of markers detected and/or indicative of the exact marker detected. Alternatively, an embodiment may produce a signal in the form of a shape that indicates the presence of one or more markers indicative of hepatitis and/or liver damage. For example, an embodiment may have a test pad with four or more test pad layers, while each layer may be sensitive to one or more markers specific to an analyte such as viral hepatitis. The respective detection of a marker in each of the test pad would generate a signal such that the detection of a marker in each of the test pad layers would confirm the diagnosis of a viral hepatitis. Although such an embodiment has been described with specific references to a viral hepatitis, it is envisioned that such an embodiment may readily be tailored to detect any number of analytes and/or markers that are specific to any analyte described below.

Embodiments may optionally possess one or more test pads and test reagents that detect analytes important to a certain age population (e.g. infants, children, young adults, adults, or elderly individuals). It is also envisioned that embodiments could possess one or more test pads and test reagents that detect analytes important to certain categories of individuals (e.g., law enforcement agents, government employers, military members, chronic drug users, physicians, veterinarians, dentists, parents, private sector employers, aid workers, inmates, hospital patients, nursing home patients, outdoorsmen, immuno-compromised individuals, or students). Embodiments may also be directed to analytes important to geographic regions (e.g. third-world countries, developed countries, or specific climate regions). Such embodiments of the invention simplify the number of different embodiments that a user must purchase or travel with because users can select embodiments that will detect the analytes the users are most interested in, or are most pertinent to a user's current or impending circumstances.

In one embodiment, a single test pad contains or has applied to it a single test reagent and/or signaling reagent suitable for detecting a single analyte. In another embodiment, two or more test pads contain or have applied to one or more of them a single test reagent and/or signaling reagent suitable for detecting a single analyte. Optionally, the single test reagent and/or signaling reagent on or applied to the two or more test pads may be the same or different. Furthermore, when different test reagents and/or signaling reagents are used, the test reagents may be sensitive to the same marker on an analyte or the test reagents may be sensitive to different markers on an analyte. The analyte may optionally be the same or different. When different analytes and different test reagents and/or signaling reagents are used, the analytes and test reagent and/or signaling reagents may be tailored to detect different symptoms of the same illness, disease, or injury. In some embodiments, a diagnosis can be made based upon the detection of all the symptoms specific to an illness, disease, or injury. In other embodiments, a diagnosis can be made based upon the absence of one or more analytes specific to an illness, disease, or injury. Using these described test pads, it is readily apparent that the reduction of false negatives and false positives can be achieved by including redundancy in the embodiments.

In one embodiment, a single test pad may contain or have applied to it two or more reagents suitable for detecting and/or signaling a single analyte. These two or more test reagents and/or signaling reagents may be sensitive to the same marker of an analyte. Optionally, these two or more reagents may be sensitive to different markers on the same analyte. In some embodiments, the two or more test reagents and/or signaling reagents may be applied to the same region of the test pad. In other embodiments, the two or more test reagents and/or signaling reagents may be applied to different regions of the same test pad. The number of test reagents and/or signaling reagents suitable for incorporation or application to a single test pad is limited only by the application of the diagnostic test strip. It is readily envisioned that embodiments may possess about one or more, about two or more, about three or more, about four or more, about five or more, about six or more, about seven or more, about eight or more, about nine or more, about ten or more, about 1-4, about 1-10, about 1-100, about 2-100, about 3-100, about 4-100, about 5-100, about 5-75, about 10-50, about 15-25, and individual numbers therein, of test reagents and/or signaling reagents incorporated or applied to one or more test pads. Using these described test pads, it is readily apparent that the reduction of false negatives and false positives can be achieved by including redundancy in the embodiments.

The one or more test pads suitable for use in an embodiment will readily detect analytes present in liquid samples, such as saliva. It is also envisioned that a test pad may be capable of detecting an analyte present in solid and/or semi-solid samples. When solid and/or semi-solid samples are analyzed, it is understood that a liquid may optionally be applied to the test pad to facilitate analysis.

When liquids and/or liquid samples are applied to test pads, lateral flow through material may result from surface tension, cohesion, adhesion, wicking, and/or capillary action. In general, embodiments that utilize lateral flow will require substantial amounts of a liquid sample for sufficient contacting of the sample with a devices test area. In some embodiments, lateral flow is confined to the test pad region. In other embodiments, lateral flow is confined to individual test pads. In further embodiments, lateral flow is confined to individual layers of a multi-layer test pad. Moreover, some embodiments overcome the use of lateral flow by having a test pad designed to absorb the fluid sample without requiring surface tension, cohesion, adhesion, wicking, and/or capillary action to contact the fluid sample with the test area. Such embodiments are particularly suited for use when the volume of a fluid sample is small and/or limited. This includes, but is not limited to, instances when the fluid sample is oral fluid such as saliva.

### Analytes

An assay based on the principles described herein can be used to determine a wide variety of analytes by choice of appropriate test reagents and/or signaling reagents. The embodiments described herein can be used to test for the existence of analytes including, but not limited to, drugs, especially drugs of abuse; heavy metals; pesticides; pollutants; proteins; polynucleotides such as DNA, RNA, rRNA, tRNA, mRNA, and siRNA; hormones; vitamins; microorganisms such as bacteria, fungi, algae, protozoa, multi-cellular parasites, and viruses; tumor markers; liver function markers; kidney function markers; blood coagulation factors; and toxins. The embodiments may also optionally detect metabolites of each of the aforementioned examples of analytes. Furthermore, some embodiments may also detect their storage conditions, specifically the temperature and humidity of their environment, and/or the application of an appropriate quantity of sample for analysis.

Analytes may be reference analytes or target analytes. Any given analyte may be either a reference analyte or a target analyte, depending upon the desired application. Indeed, any analyte described below that is known to consistently be present in a given sample may serve as a reference analyte. As a non-limiting example, alpha-amylase is an enzyme present in saliva and could serve as a reference analyte when the analyzed sample is saliva. However, methadone could serve as a reference analyte when an embodiment is desired for use with samples obtained from patients generally known and/or suspected of having methadone in their system. Thus, one will readily appreciate that it is the application of the embodiment that determines the analytes classified as references or targets.

More specific examples of drug analytes, including both drugs of abuse and therapeutic drugs, include benzheterocyclics, the heterocyclic rings being azepines, diazepines and phenothiazines. Examples of azepines include fenoldopam. Examples of benzodiazepines include alprazolam, bretazenil, bromazepam, chlorodiazepoxide, cinolazepam, clonazepam, cloxazolam, clorazepate, diazepam, estazolam, fludiazepam, flunirazepam, flurazepam, flutoprazepam, halazepam, ketazolam, loprazolam, lorazepam, lormetazepam, medazepam, midazolam, nimetazepam, nitrazepam, nordazepam, oxazepam, phenazepam, pinazepam, prazepam, premazepam, quazepam, temazepam, tetrazepam, triazolam, and other benzodiazepine receptor ligands such as clobazam, DMCM, flumazenil, eszopiclone, zaleplon, zolpidem, and zopiclone. Examples of phenothiazines include chlorpromazine, promethazine, triflupromazine, methotrimeprazine, mesoridazine, thioridazine, fluphenazine, perphenazine, prochlorperazine, and trifluoperazine. Examples of other benzheterocyclics include, but are not limited to, carbamazepine and imipramine.

Additional drug analytes, including both drugs of abuse and therapeutic drugs, include alkaloids, such as agents that interact with opioid receptors including morphine, dihydromorphine, desomorphine, hydromorphone, nicomorphine, oxymorphone, hydromorphinol, nalbuphine, naloxone, naltrexone, buprenorphine, etorphine, metopon, diacetyldihydromorphine, thebacon, methodone, codeine, hydrocodone, dihydrocodeine, oxycodone, papaveretum, oripavine, thebaine, tapentadol, and heroin; agents that exert effects on serotonin receptors, such as cocaine (and other reuptake inhibitors, including norepinephrine, dopamine, and serotonin reuptake inhibitors); cocaine metabolites such as benzoylecgonine; ergot alkaloids; steroid alkaloids; iminazoyl alkaloids; quinazoline alkaloids; isoquinoline alkaloids; quinoline alkaloids; and diterpene alkaloids.

Another group of drug analytes, including both drugs of abuse and therapeutic drugs, includes steroids, including the estrogens, gestogens, androgens, andrenocortical steroids, bile acids, cardiotonic glycosides and aglycones, which includes digoxin and digoxigenin, saponins and sapogenins, their derivatives and metabolites.

Additional drug analytes, including both drugs of abuse and therapeutic drugs, is the barbiturates, such as barbital, allobarbital, amobarbital, aprobarbital, alphenal, brallobarbital, Phenobarbital, pentobarbital, Nembutal, secobarbital, diphenylhydantonin, primidone, and ethosuximide. Additionally, drugs similar in effect to barbiturates are potential analytes, such as methaqualone, cloroqualone, diproqualone, etaqualone, mebroqualone, mecloqualone, methylmethaqualone, and nitromethaqualone.

Another group of drug analytes, including both drugs of abuse and therapeutic drugs, is aminoalkylbenzenes, including the phenethylamines such as amphetamine, methamphetamine, lisdexamfetamine, mescaline, and catecholamines, which includes ephedrine, L-dopa, epinephrine, narceine, and papaverine.

Additional drug analytes, including both drugs of abuse and therapeutic drugs, includes those derived from marijuana, which includes cannabinol, tetrahydrocannabinol, 11-nor-9-carboxy-delta-9-tetrahydrocannabinol, nabilone, dronabinol, marinol, and cannabinoids such as cannabidiol, cannabinol, and tetrahydrocannabivarin.

Another group of drug analytes, including both drugs of abuse and therapeutic drugs, are those that interact with the N-methyl d-aspartate (“NMDA”) receptor, including agonists, modulators, and antagonists such as 1-(1-phylcyclohexyl)piperidine (phencyclidine or “PCP”), R-2-amino-5-phosphonopentanoate, 2-amino-7-phosphonoheptanoic acid, (3-[(R)-2-carboxypiperazin-4-yl]-prop-2-enyl-1-pho phonic acid), PEAQX, selfotel, amantadine, dextrallorphan, dextromethorphan, dextrorphan, dizocilpine, ethanol, eticyclidine, gacyclidine, ibogaine, ketamine, memantine, methoxetamine, rolicyclidine, tenocyclidine, tiletamine, neramexane, eliprodil, etoxadrol, dexoxadrol, NEFA, remacemide, delucemine, 8A-PDHQ, aptiganel, HU-211, remacemide, atomoxetine, rhynchophylline, 1-aminocyclopropanecarboxylic acid, 7-chlorokynurenate, 5,7-dichlorokynurenic acid, kynurenic acid, and lacosamide.

Another group of therapeutic drugs is antibiotics, which include, for example, beta-lactam antiobiotics such as penicillins and cephalosporins, penems and carbapenems, antimicrobials such as aminoglycosides, ansamycins, carbacephems, glycopeptides, lincosamides, lipopetides, macrolides, monobactams, nitrofurans, quionolones, polypeptide-based antibiotics, chloromycetin, actinomycetin, spectinomycin, sulphonamides, trimethoprim, tetracyclines, and beta-lactamase inhibitors such as calvulanic acid, tazobactam, and sulbactam.

Other individual miscellaneous drug analytes, including both drugs of abuse and therapeutic drugs, include nicotine, caffeine, gamma-hydroxybutyric acid, dextromoramide, ketobemidone, piritramide, dipipanone, phenadoxone, benzylmorphine, nicocodeine, dihydrocodeinone enol acetate, tilidine, meptazinol, propiram, acetyldihydrocodeine, pholcodine, 3,4-methylenedioxymethamphetamine, psilocybin, 5-methoxy-N,N-diisopropyltryptamine, peyote, 2,5-dimethoxy-4-methylamphetamine, 2C-T-7 (a psychotropic entheogen), 2C-B, cathinone, alpha-methyltryptamine, bufotenin, benzylpiperazine, methylphenidate, dexmethylphenidate, laudanum, fentanyl, mixed amphetamine salts (i.e. Adderall), lisdexamfetamine, dextroamphetamine, dextromethamphetamine, pethidine, anabolic steroids, talbutal, butalbital, buprenorphine, xyrem, paregoric, modafinil, difenoxin, diphenoxylate, promethazine, pregabaline, pyrovalerone, atropine, and other Schedule I-V classified drugs, glucose, cholesterol, bile acids, fructosamine, carbohydrates, metals which includes, but is not limited to lead and arsenic, alcohols (i.e. methanol, ethanol, propanol, butanol, and C₅₋₁₀ containing alcohols), meprobamate, serotonin, meperidine, amitriptyline, nortriptyline, lidocaine, procaineamide, acetylprocainearnide, propranolol, griseofulvin, valproic acid, butyrophenones, antihistamines, and anticholinergic drugs, such as atropine.

Pesticide analytes of interest include categories such as algicides, avicides, bactericides, fungicides, herbicides, insecticides, miticides, molluscicides, nematicides, rodenticides, virucides, and specifically polyhalogenated biphenyls, phosphate esters, thiophosphates, carbamates, and polyhalogenated sulfenamides.

Additional chemical analytes of interest include fertilizers such as ammonium derivatives, nitrates, and phosphates; heavy metals such as lead, mercury, uranium, plutonium, arsenic, cadmium, chromium, and nickel

More specific examples of protein analytes include antibodies, protamines, histones, albumins, globulins, scleroproteins, phosphoproteins, mucoproteins, chromoproteins, lipoproteins, nucleoproteins, glycoproteins, proteoglycans, and unclassified proteins, such as somatotropin, prolactin, insulin, and pepsin. A number of proteins found in the human plasma are important clinically and include prealbumin, albumin, α₁-lipoprotein, α₁-acid glycoprotein, α₁-antitrypsin, α₁-glycoprotein, transcortin, 4.6S -postalbumin, tryptophan-poor, α₁-glycoprotein, α₁X-glycoprotein, thyroxin-binding globulin, inter-α-trypsin-inhibitor, Gc-globulin (Gc I-I, Gc 2-1, Gc 2-2), haptoglobin, ceruloplasmin, cholinesterase, α₂-lipoprotein(s), myoglobin, C-reactive Protein, α₂-macroglobulin, α₂-HS-glycoprotein, Zn-α₂-glycoprotein, α₂-neuramino-glycoprotein, erythropoietin, β-lipoprotein, transferrin, hemopexin, fibrinogen, plasminogen, β₂-glycoprotein I, β₂-glycoprotein II, immunoglobulins A, D, E, G, M, prothrombin, thrombin, and protein markers in cancers including, but not limited to, breast cancer, prostate cancer, melanoma, carcinoma, pancreatic cancer, liver cancer, and brain cancer.

Additional protein analytes of interest include alanine aminotransferase and aspartate aminotransferase. Alanine aminotransferase is markedly elevated when hepatitis is present in the liver. Such elevation for alanine aminotransferase may include at least about 1.25, 1.5, 1.75, 2, 2.25, 2.5, 2.75, and 3.0 times the normal levels associated with a person lacking liver damage. Aspartate aminotransferase is elevated when cellular damage occurs, such as liver damage, skeletal muscle damage, and acute myocardial infarction. Additionally, levels are elevated because of congestive heart failure, pericarditis, cirrhosis, metastatic liver disease, skeletal muscle diseases, and generalized infections such as mononucleosis. Such elevation for aspartate aminotransferase may include at least about 1.25, 1.5, 1.75, 2, 2.25, 2.5, 2.75, and 3.0 times the normal levels associated with a person lacking liver damage. Consequently, the detection of alanine aminotransferase and/or aspartate aminotransferase is of therapeutic importance.

Specific examples of peptide and protein hormone analytes include parathyroid hormone (parathromone), thyrocalcitonin, insulin, glucagon, relaxin, erythropoietin, melanotropin (melanocyte-stimulating hormone and intermedin), somatotropin (growth hormone), corticotropin (adrenocorticotropic hormone), thyrotropin, prolactin, follicle-stimulating hormone, luteinizing hormone), chorionic gonadotropin (hCG), oxytocin, and vasopressin.

Specific examples of polynucleotide analytes include DNA and RNA as well as their nucleoside and nucleotide precursors, which include ATP, NAD, FMN, adenosine, guanosine, thymidine, cytidine, and uracil with their appropriate sugar and phosphate substituents.

Specific examples of vitamin analytes include Vitamin A (i.e. retinol), B (e.g. B₁ or thiamine, B₂ or riboflavin, B₃ or niacin, B₅ or pantothenate, B₆ or pyridoxine, B₇ or biotin, B₉ or folic acid, and B₁₂), C (i.e. ascorbic acid), D (e.g. calciferol, D₂, and D₃), E (i.e. tocopherol), K, and vitamin derivatives or metabolites such as nicotinamide.

Specific examples of microorganism analytes, including infectious disease agents, include corynebacteria, pneumococci, streptococci, staphylococci, neisseriae, hemophilus influenzae, pasteurellae, brucellae, aerobic spore-forming bacilli, anaerobic spore-forming bacilli, mycobacteria, actinomycetes (fungus-like bacteria), the spirochetes, mycoplasmas, and other pathogens, such as listeria monocytogenes, erysipelothrix rhusiopathiae, streptobacillus moniliformis, donvania granulomatis, bartonella bacilliformis, rickettsiae (bacteria-like parasites), fungi, agents causing venereal diseases such as chlamydia, chancroid, granuloma inguinale, gonorrhea, syphilis, jock itch, yeast infection, herpes simplex, HPV, crab louse, scabies, trichomoniasis, and infectious diarrheal microorganisms such as camplylobacter, salmonellae, shigellae, Escherichia coli, Clostridium difficile, Giardia lamblia, Entamoeba histolytica, and organisms causing leptospirosis, nosocomial infections, staphylococcal enterotoxicosis, typhoid fever, cholera, vibrio gastroenteritis, yersinia gastroenteritis, clostridium perfringens gastroenteritis, bacillus cereus gastroenteritis, aflatoxin poisoning, amoebic dysentery, cryptosporidiosis, cyclospora diarrheal infection. Other microorganism analytes include viruses, such as herpes viruses, pox viruses, picornaviruses, myxoviruses (influenza A, B, and C, and mumps, measles, rubella, etc.), arboviruses, reoviruses, rotoviruses, noroviruses, adenoviruses, astroviruses, hepatitis, human immunodeficiency virus, and tumor viruses.

The categories of protein analytes and microorganism analytes may optionally overlap. For example, a microorganism analyte may be detected via the analysis of a protein analyte specific for the microorganism analyte. A protein analyte specific for a microorganism analyte may include an antibody specific for a microorganism analyte, or marker thereof. As a non-limiting example, for a microorganism analyte such as viral hepatitis, antibodies specific to any of viral hepatitis A, B, C, D, E, F and/or G may comprise the protein analyte. Such antibodies include, but are not limited to, immunoglobins such as IgA, IgD, IgE, and specifically IgM and/or IgG, and antibodies to surface antigens, envelope antigens, core antigens, and/or delta antigens (e.g. small and/or large). Specific examples of antigens for viral hepatitis B include hepatitis B surface antigen (HBsAg), hepatitis B envelope antigen (HBeAg), hepatitis B core antigen (HBcAg). Alternatively, a protein analyte specific for a microorganism analyte may include a protein analyte characteristically produced by the microorganism analyte. As a non-limiting example, for a microorganism analyte such as viral hepatitis, proteins specific to any of viral hepatitis A, B, C, D, E, and/or F may comprise the protein analyte. Such protein analytes include, but are not limited to, structural and/or nonstructural proteins. Specific examples of protein analytes for viral hepatitis C include, but are not limited to structural proteins such as E1 and/or E2, and/or nonstructural proteins such as NS2, NS3, NS4, NS4A, NS4B, NS5, NS5A, NS5B, and peptide portions thereof.

The above described analytes possess at least one marker recognized by at least one test reagent and/or signaling reagent. Optionally, the above described analytes may possess multiple markers recognized by the same and/or different test reagents and/or signaling reagents. It is readily envisioned that a marker may be the entire analyte and/or a portion thereof.

### Samples

An analyte of interest may be present in a wide variety of environments, and it is envisioned that a person having ordinary skill in the art will readily understand that the components and embodiments discussed above can be modified as needed to accommodate different environments of samples.

Analytes of interest may be found in a patient's physiological fluids, such as mucus, blood, serum, blood plasma, lymph, puss, urine, feces, cerebral spinal fluid, ocular lens liquid, ascites, semen, sputum, saliva, sweat, and secreted oils. Samples for testing analytes may be obtained using techniques known or envisioned to provide samples of such physiological fluids. Optionally, analytes may be detected by directly contacting embodiments of the diagnostic test strips with the patient's body, such as their skin, eyes, mouth cavity regions including the tongue, tonsils, and inner lining of the mouth and throat, and the nasal cavity. Alternatively, some analytes may be detected by directly contacting embodiments of the diagnostic test strips with a patient's urine stream, source of bleeding, source of puss, discharge from sex organs, or other site of fluid leakage from the patient.

Analytes may also be found in synthetic chemicals, water, soil, air and food (e.g., milk, meat, poultry, or fish). Any organic- and inorganic-containing substances can serve as an analyte so long as test reagents are available to generate a signal concerning the presence, absence, and/or concentration of the analyte.

For oral fluids such as saliva, samples may be obtained by contacting an embodiment with a patient's tongue such that the tongue contacts the one or more test pads. Alternatively, salivary samples may be obtained by contacting an embodiment with the top and/or sides of a patient's tongue using a substantially back and forth motion from substantially the tip of the tongue to substantially the back of the tongue. Furthermore, salivary samples may be obtained by contacting an embodiment with the top and/or sides of a patient's tongue using a substantially side-to-side motion along the width of the tongue. Similarly, salivary samples may also be obtained by contacting an embodiment with the top and/or sides of a patient's tongue using a substantially circular motion. For each of the above described sample collection methods, the results of the analysis could then be read directly from the diagnostic test strip by a user. Optionally, test results could be stored to a suitable memory device for recordation and later access.

Prior to use with embodiments of the invention, samples may be preserved, stored, or pre-treated in manners consistent with known handling of the same, or similar, types of samples. It is envisioned that any type of preservation, storage, or pre-treatment may be utilized so long as it does not introduce false positives or false negatives into the assay.

### CONCLUSION

While the invention has been described with reference to the specific embodiments thereof, it should be understood by those skilled in the art that various changes may be made and equivalents may be substituted without departing from the true spirit and scope of the invention. For example, some embodiments do not provide all of the benefits and features set forth herein. In addition, many modifications may be made to adapt a particular situation, material, composition of matter, process, process step or steps, to the objective, spirit and scope of the present invention. Furthermore, practiced embodiments may include features of more than one of the described embodiments. All such modifications are intended to be within the scope of the claims appended hereto. Accordingly, the scope of the invention is defined only by reference to the appended claims.

What is claimed is:

1. An apparatus that is a diagnostic assay test strip comprising one or more indentations wherein the each of the indentations contains at least one test pad, said test pad having a top and a bottom surface, wherein the bottom surface is in contact with the test strip.

2. The test strip of claim 1, wherein the test pad contains one or more test reagents.

3. The test strip of claim 1, wherein each of the one or more indentations extend across the width of the test strip

4. The test strip of claim 1, wherein there is a single indentation.

5. The test strip of claim 1, wherein there are two indentations.

6. The test strip of claim 1, wherein the indentation extends for substantially the entire width of the test strip.

7. The test strip of claim 1, wherein the top surface of the test pad is flush with the surface of the test strip.

8. The test strip of claim 1, wherein the top surface of the test pad is below the surface of the test strip.

9. The test strip of claim 1, wherein the indentations are sealed with a removable cover.

10. A diagnostic test strip comprising a carrier strip and at least two test pads, wherein at least one test pad is disposed on the opposing side of the carrier strip from at least one other test pad and wherein the test pads extend above the surface of the carrier strip.

11. The diagnostic test strip of claim 10, wherein at least one test pad contains a test reagent.

12. The diagnostic test strip of claim 10, wherein each of the at least two test pads contain test reagents and the test reagents in each pad are different.

13. The diagnostic test strip of claim 12, wherein the test reagents each test for a different marker on the same analyte.

14. The diagnostic test strip of claim 10, wherein at least one test pad contains a signaling reagent.

15. The diagnostic test strip of claim 10, wherein the carrier strip is substantially non-porous.

16. The diagnostic test strip of claim 10, wherein the carrier strip is substantially porous.

17. The diagnostic test strip of claim 10, further comprising one or more additional test pads disposed on the opposing or adjacent sides of the carrier strip.

18. The diagnostic test strip of claim 10, wherein the test pads are substantially porous.

19. The diagnostic test strip of claim 10, wherein the test pads are substantially non-porous.

20. The diagnostic test strip of claim 10, wherein the at least two test pads extend for substantially the entire width of the side of the strip to which they are disposed.

21. The test strip of claim 10, wherein at least one test pad has a test reagent that tests for an analyte found in saliva.

22. The test strip of claim 10, wherein at least one test pad has a test reagent that tests for an analyte found in serum.

23. The test strip of claim 10, wherein at least one test pad has a test reagent that tests for an analyte found in plasma.

24. The test strip of claim 10, wherein at least one test pad has a test reagent that tests for an analyte found in urine.

25. The test strip of claim 10, wherein at least one test pad has a test reagent that tests for an analyte found in blood.

26. The test strip of claim 10, wherein at least one test pad has a test reagent that tests for an analyte found in semen.

27. The test strip of claim 10, wherein at least one test pad has a test reagent that tests for an analyte found in sputum.

28. The test strip of claim 10, wherein at least one test pad has a test reagent that tests for an analyte found in cerebral spinal fluid.

29. The test strip of claim 10, wherein at least one test pad has a test reagent that tests for an analyte found in ascites.

30. The test strip of claim 10, wherein at least one test pad has a signal reagent for an analyte.

31. A method of detecting one or more analytes in a patient sample, comprising:
a) acting the test strip of claim 1 with the patient sample such that the test pads come in contact with the sample; and
b) reading the test result from the one or more test pads.

32. The method of claim 31, further comprising adding one or more signaling reagents to the test strip following the addition of the patient sample such that the reagents contacts the test pads.

33. The method of claim 31, wherein the patient sample is saliva.

34. The method of claim 33, wherein the test strip is contact with the patient's tongue.

35. The method of claim 31, wherein the patient sample is serum.

36. The method of claim 31, wherein the patient sample is semen.

37. The method of claim 31, wherein the patient sample is blood.

38. The method of claim 37, wherein the test strip directly contracts the source of the blood on the patient.

39. The method of claim 31, wherein the patient sample is plasma.

40. The method of claim 31, wherein the patient sample is ascites.

41. The method of claim 31, wherein the patient sample is sputum.

42. The method of claim 31, wherein the patient sample is cerebral spinal fluid.

43. A diagnostic test strip comprising:
a) a test pad carrier,
b) one or more test pads, where the test pads are interspersed, and interrupt, the test pad carrier,
c) an intermediate laminate layer, where the intermediate layer is longer than the test pad carrier, and the test pad carrier and test pads are disposed on top of the intermediate layer, and
d) a bottom laminate layer, where the bottom of the intermediate layer is disposed on top of the bottom laminate layer,
wherein at least one of the test pads contains a test reagent.

44. The diagnostic test strip of claim 43, wherein the top of the test pad carrier and the one or more test pads are substantially flush.

45. The diagnostic test strip of claim 43, wherein the one or more test pads extends above the top of the test pad carrier.

46. The diagnostic test strip of claim 43, wherein there is one test pad.

47. The diagnostic test strip of claim 43, wherein one test pad is at one end of the test strip.

48. The diagnostic test strip of claim 43, wherein there are at least two test pads.

49. The diagnostic test strip of claim 48, wherein each test pad contains a different test reagent.

50. The diagnostic test strip of claim 48, wherein the two test pads are separated by a length of test carrier pad.

51. The diagnostic test strip of claim 43, wherein there are more than one test pad and one test pad does not contain a test reagent.

52. The diagnostic test strip of claim 43, wherein the at least one test pad contain a reagent that tests for an analyte present in mammalian saliva.

53. The diagnostic test strip of claim 43, wherein the at least one test pad contain a reagent that tests for an analyte present in mammalian urine.

54. The diagnostic test strip of claim 43, wherein the at least one test pad contain a reagent that test for an analyte present in plasma.

55. The diagnostic test strip of claim 43, wherein the at least one test pad contain a reagent that tests for an analyte present in serum.

56. The diagnostic test strip of claim 43, wherein the at least one test pad contains a reagent that tests for an analyte present in blood.

57. The diagnostic test strip of claim 43, wherein the at least one test pad contains a test reagent that tests for an analyte present in ascites.

58. The diagnostic test strip of claim 43, wherein the at least one test pad contains a reagent that tests for an analyte present in cerebral spinal fluid.

59. The diagnostic test strip of claim 43, wherein the at least one test pad contains a reagent that tests for an analyte present in semen.

60. The diagnostic test strip of claim 43, wherein the at least one test pad contains a reagent that tests for an analyte present in sputum.

61. The diagnostic test strip of claim 43, wherein a signal generating reagent is present on the at least one test pad.

62. The diagnostic test strip of claim 61, wherein a signal generating reagent is on the test pad containing a test reagent.

63. The diagnostic test strip of claim 43, wherein there are more than one test pads and each one detects a different marker on the same analyte.

64. A method for detecting one or more analytes in a patient sample, comprising:
1) applying the sample on the test strip of claim 43 so that it contacts the one or more test pads present on the test strip; and
2) reading the test result from the one or more test pads.

65. The method of claim 64, further comprising removing any removable covers present on the test strip before applying the sample.

66. The method of claim 64, wherein the patient sample applied is saliva.

67. The method of claim 64, wherein the patient sample is applied by contacting the test strip with patient's tongue.

68. The method of claim 64, further comprising applying a signal developing reagent to the test strip prior to reading the test result.

69. The method of claim 64, wherein the patient sample is urine.

70. The method of claim 69, wherein the patient applies the urine directly to the test strip.

71. The method of claim 64, wherein the patient sample is plasma.

72. The method of claim 64, wherein the patient sample is serum.

73. The method of claim 64, wherein the patient sample is blood.

74. The method of claim 64, wherein the test strip is directly contacted with the source of the blood sample from the patient.

75. The method of claim 74, wherein the patient sample is semen.

76. The method of claim 64, wherein the patient sample is ascites.

77. The method of claim 64, wherein the patient sample is sputum.

78. The method of claim 64, wherein the patient sample is cerebral spinal fluid.

79. The method of claim 64, wherein at least one signaling reagent is added to the test strip such that it contacts the one or more test pads.

80. A diagnostic test strip having a top, bottom and sides wherein one or more test pads comprised of a top, bottom and sides, further wherein the top and the bottom of the pads have a trailing edge and a leading edge are mechanically fixed to the test strip.

81. The diagnostic test strip of claim 80, wherein the test pads are fixed to the test strip with staples.

82. The diagnostic test strip of claim 81, wherein a one or more of a first group of staples bridges the trailing edge and the test strip and wherein one or more of a second group of staples brides the leading edge of the test pad and the test strip.

83. The diagnostic test strip of claim 82, wherein one staple each bridges the trailing and leading edges of the test pads and the test strip.

84. The diagnostic test strip of claim 82, further wherein one or more of a third group of staples bridge one side of the test pad and the test strip and one or more staples of a fourth group of staples bridges the side of test pad and the side and the top of the test strip.

85. The diagnostic test strip of claim 81, wherein there are at least two or more test pads and at least one is attached to the top of the test strip and at least one is attached to the bottom of the test strip.

86. The diagnostic test strip of claim 81, wherein there are at least two or more test pads and at least one is attached to the top of the test strip and at least one other is attached to a side of the test strip.

87. The diagnostic test strip of claim 80, wherein each of the test pads contains a test reagent.

88. The diagnostic test strip of claim 80, wherein at least one of the test pads further contains a signaling reagent.

89. The diagnostic test strip of claim 80, wherein each of the test pads contains a different test reagent.

90. The diagnostic test strip of claim 80, wherein each of the test pads has a different test reagent and each of the reagent detects a different marker on the same analyte.

91. The diagnostic test strip of claim 80, wherein a single test pad is mechanically fixed to the top of the pad.

92. The diagnostic test strip of claim 80, wherein two test pads are mechanically fixed to the top of the pad.

93. The diagnostic test strip of claim 80, wherein three test pads are mechanically fixed to the top of the pad.

94. The diagnostic test strip of claim 80, further wherein at least one test pad is mechanically fixed to the top of the strip and at least one test pad is attached test pad to the bottom of the strip.

95. The test strip of claim 94, further wherein one test pad is mechanically attached to the top of the test strip and one mechanically attached to a side of the test strip.

96. The diagnostic test strip of claim 81, wherein the staples are metal.

97. The diagnostic test strip of claim 81, wherein the staples are paper.

98. The diagnostic test strip of claim 81, wherein the staples are plastic.

99. The diagnostic test strip of claim 80, wherein the test strip has indentations in the top of the strip and the test pads are fixed to the strip in these indentations.

100. The diagnostic test strip of claim 99, wherein the indentations contain the entire depth of the test pad such that the top of the test pad is flush with the top of the test strip.

101. The diagnostic test strip of claim 99, wherein the indentations are such that the top of the test pads are below the top of the test strip.

102. The diagnostic test strip of claim 80, wherein the test pads extend substantially from side to side on the top of the test strip.

103. The diagnostic test strip of claim 80, wherein the test strip is substantially porous.

104. The diagnostic test strip of claim 80, wherein the test strip is substantially non-porous.

105. The diagnostic test strip of claim 80, wherein the test pads are substantially porous.

106. The diagnostic test strip of claim 80, wherein the test pads are substantially non-porous.

107. The diagnostic test strip of claim 80, wherein the at least one test pad has a reagent that tests for a saliva-borne analyte.

108. The diagnostic test strip of claim 80, wherein the at least one test pad has a reagent that tests for a sputum-borne analyte.

109. The diagnostic test strip of claim 80, wherein the at least one test pad has a reagent that tests for a serum-borne analyte.

110. The diagnostic test strip of claim 80, wherein the at least one test pad has a reagent that tests for a plasma-borne analyte.

111. The diagnostic test strip of claim 80, wherein the at least one test pad has a reagent that tests for a blood-borne analyte.

112. The diagnostic test strip of claim 80, wherein the at least one test pad has a reagent that tests for a urine-borne analyte.

113. The diagnostic test strip of claim 80, wherein the at least one test pad has a reagent that tests for a semen-borne analyte.

114. The diagnostic test strip of claim 80, wherein the at least one test pad has a reagent that tests for a cerebral spinal fluid-borne analyte.

115. The diagnostic test strip of claim 80, wherein the at least one test pad has a reagent that tests for an ascites-borne analyte.

116. A method for detecting analytes in a patient sample, comprising:
a) contacting the test strip of claim 80 with a patient sample so that the sample contacts the one or more test pads; and
b) reading the results from the test strip.

117. The method of claim 116, further comprising contacting the test strip with one or more signaling reagents such that the one or more signaling reagents contacts the one or more test pads.

118. The method of claim 116, wherein the patient sample is serum.

119. The method of claim 116, wherein the patient sample is semen.

120. The method of claim 116, wherein the patient sample is urine.

121. The method of claim 121, wherein the test strip is directly contacted with the patient's urine stream.

122. The method of claim 116, wherein the patient sample is saliva.

123. The method of claim 122, wherein the test strip is contacted with patient's tongue.

124. The method of claim 116, wherein the patient sample is blood.

125. The method of claim 124, wherein the test strip is contacted directly with the source of the blood.

126. The method of claim 116, wherein the patient sample is ascites.

127. The method of claim 116, wherein the patient sample is sputum.

128. The method of claim 116, wherein the patient sample is cerebral spinal fluid.
